(19) United States
(12) Patent Application Publication
(10) Pub. No.: US 2021/0401291 A1
(43) Pub. Date: Dec. 30, 2021

(54) Apparatus and Method for Analyzing a Substance

(71) Applicant: DiaMonTech AG, Berlin (DE)

(72) Inventors: Uwe Schriek, Berlin (DE); Wilfried Noell, Neuchatel (CH); Thorsten Lubinski, Berlin (DE)

(21) Appl. No.: 17/290,563

(22) PCT Filed: Jun. 3, 2019
(86) PCT No.: PCT/EP2019/064356

§ 371 Date: Apr. 30, 2021

(30) Foreign Application Priority Data
Nov. 8, 2018 (EP) PCT/EP2018/080665

(51) Int. Cl. A61B 5/00; G16H 10/40; G16H 50/20; A61B 5/01; A61B 5/145

(52) U.S. Cl. CPC A61B 5/0075; G16H 10/40; G16H 50/20; A61B 5/0022; A61B 2562/028; A61B 5/01; A61B 5/14532; A61B 5/6826; A61B 2562/0238; A61B 5/0095

(57) ABSTRACT

The invention relates to a device for analyzing a substance, comprising: —a measurement body (1, 1a), which has a measurement surface (2) and is to be brought at least in part into contact with the substance (3) in the region of the measurement surface for the purpose of measuring; a laser device (4), particularly having a quantum cascade laser (QCL), a tunable QCL and/or a laser array, preferably an array of QCLs, in order to generate one or more excitation beams (10) at different wavelengths, preferably in the infrared or medium infrared spectral range, which is directed to the substance (3); and a detection apparatus (5, 6, 7) which is integrated at least in part in the measurement body (1, 1a) or connected thereto and comprises the following: •a source (5) for coherent detection light (11) and •a first optical waveguide structure (6) which can be or is connected to the source for the detection light, which guides the detection light, and has a refractive index which is dependent at least in portions on the temperature and/or pressure, wherein the first optical waveguide structure has at least one portion (9) in which the light intensity depends on a phase shift of detection light in at least one part of the first optical waveguide structure (6) due to a change in temperature or pressure.

The present property right relates to an apparatus and a method for analysing a substance. These can be used, for example, for the analysis of animal or human tissue, fluids, in particular bodily fluids and in one embodiment, for measuring glucose or blood sugar.

Known methods for analysing a substance, in particular for measuring blood sugar, are described, for example, in the following documents:
1. Guo et al.: “Noninvasive glucose detection in human skin using wavelength modulated differential laser photothermal radiometry”, Biomedical Optics Express, Vol. 3, 2012, No. 11,
2. Uemura et al.: “Non-invasive blood glucose measurement by Fourier transform infrared spectroscopic analysis through the mucous membrane of the lip: application of a chalcogenide optical fiber System”, Front Med Biol Eng. 1999; 9(2): 137-153,
3. Farahi et al.: “Pump probe photothermal spectroscopy using quantum cascade lasers”, J. Phys. D. Appl. Phys. 45 (2012) and
4. M. Fujinami et al.: “Highly sensitive detection of molecules at the liquid/liquid interface using total internal reflection-optical beam deflection based on photothermal spectroscopy”, Rev. Sci. Instrum., Vol. 74, Number 1 (2003).
5. von Lilienfeld-Toal, H. Weidenmüller, M. Xhelaj, A. Mäntele, W. A Novel Approach to Non-Invasive Glucose Measurement by Mid-Infrared Spectroscopy: The Combination of Quantum Cascade Lasers (QCL) and Photoacoustic Detection Vibrational Spectroscopy, 38:209-215, 2005.
6. Pleitez, M. von Lilienfeld-Toal, H. Mäntele W. Infrared spectroscopic analysis of human interstitial fluid in vitro and in vivo using FT-IR spectroscopy and pulsed quantum cascade lasers (QCL): Establishing a new approach to non-invasive glucose measurement. Spectrochimica Acta. Part A, Molecular and biomolecular spectroscopy, 85:61-65, 2012
7. Pleitez, M. et al. In Vivo Noninvasive Monitoring of Glucose Concentration in Human Epidermis by Mid-Infrared Pulsed Photoacoustic Spectroscopy Analytical Chemistry, 85:1013-1020, 2013.
8. Pleitez, M. Lieblein, T. Bauer, A. Hertzberg, O. von Lilienfeld-Toal, H. Mäntele, W. Windowless ultrasound photoacoustic cell for in vivo mid-IR spectroscopy of human epidermis: Low interference by changes of air pressure, temperature, and humidity caused by skin contact opens the possibility for a non-invasive monitoring of glucose in the interstitial fluid. Review of Scientific Instruments 84, 2013
9. M. A. Pleitez Rafael, O. Hertzberg, A. Bauer, M. Seeger, T. Lieblein, H. von Lilienfeld-Toal, and W. Mäntele. Photothermal deflectometry enhanced by total internal reflection enables non-invasive glucose monitoring in human epidermis. The Analyst, November 2014.

The object of the invention is to provide an apparatus and a method which can be used to analyse a substance, in particular an animal or human tissue or a component or constituent of the tissue, or a fluid, in a particularly simple, accurate and cost-effective manner. One aspect of the invention also involves the achievement of a small size of the apparatus.

In addition, reference is made to the German patent document DE 10 2014 108 424 B3.

This object is achieved, inter alia, by an apparatus having the features in accordance with claim 1. Embodiments of the apparatus are specified in sub-claims. In addition, the invention relates to a method in accordance with the independent method claim with corresponding embodiments according to the sub-claim(s) dependent thereon.

In addition to the subject matter of the claims and exemplary embodiments explicitly mentioned at the time of filing, this patent application also refers to other aspects listed at the end of the present description. These aspects can be combined individually or in groups, in each case with features of the claims cited at the time of filing. These aspects also constitute independent inventions, whether taken in isolation or combined with one another or with the claimed subject matter of this application. The applicant reserves the right to make these inventions the subject of claims at a later date. This may occur as part of this application or within the context of subsequent divisional applications, continuation applications (U.S.), continuation-in-part applications (U.S.), or subsequent applications that claim priority of this application.

In connection with the following description the terms “light” or “laser light” mean electromagnetic waves or electromagnetic radiation in the visible range, the near, medium and far infrared range, and in the UV range.

The following text first deals with the subject matter of the claims listed at the time of filing.

The object is achieved with the features of the invention according to patent claim 1 by a device for analysing a substance having:
a measuring body which has a measuring surface and is to be at least partially coupled with the substance in the area of the measuring surface for measurement, in particular directly or by means of a medium, in particular a fluid, or is to be brought into contact with it directly or else by means of a medium,
a source of excitation radiation capable of generating light or an excitation beam of different wavelengths, in particular a laser device, in particular with a quantum cascade laser (QCL), a tuneable QCL, and/or with a laser array, preferably an array of QCLs, for generating one or more excitation beams with different wavelengths, preferably in the infrared spectral range, which is directed at the substance when the measuring body is coupled and/or in contact with the substance in the region of the measuring surface, and
a detection device, which is at least partially integrated into the measuring body or connected to it, comprising the following:
   a source for detection light, preferably coherent detection light, and
   a first optical waveguide structure, which can be or is connected to the detection light source and which guides the detection light, the refractive index of which, at least in some sections, is dependent on the temperature and/or pressure, the first optical waveguide structure having at least one section in which the light intensity depends on a phase shift of detection light in at least one part of the optical waveguide structure due to a change in temperature or pressure.

In this context, a phase shift of the detection light is understood to mean a phase shift relative to the phase position of the detection light before or without the temperature or pressure change. A phase shift of the detection light can thus be determined from the change in the light intensity, and from this a change in the refractive index. From the change in the refractive index, for example, the intensity of a thermal and/or pressure wave can be determined, from which in turn, in preferred embodiments, an absorption strength can be determined and from this the concentration of a substance to be detected. In addition to visible light, the term detection light can also mean infrared or UV light or another type of electromagnetic waves that can be passed through the optical waveguide structure.

Energy is injected into the substance by means of the excitation beams and the excitation beams are absorbed to a greater or lesser extent as a function of the irradiated light wavelength and the substances present in the substance to be analysed as well as their resonance vibrations or absorption frequencies, wherein heat energy is released in the form of molecular vibrations. In addition to a tuneable laser or a laser array, the wavelength-tuneable light source can also be formed by a different type of radiation source, e.g. a broadband light source from which individual wavelengths can be selected optionally by filters. For example, one or more light emitting diodes in the infrared range can be used, the radiation of which can be selected in narrow bands over desired wavelength ranges. Here also, modulation can take place in the light source or in the optical path.

In terms of its intensity, the heating process follows the modulation of the excitation beam and generates a thermal and/or pressure wave which propagates in the substance to be analysed, inter alia, towards and also in the measuring body and influences the first optical waveguide structure in the detection device. The measuring body is coupled with the substance in the area of the measuring surface, so that a thermal and/or pressure wave can pass from the substance onto the measuring body. The coupling can take place directly through physical contact between the substance and the measuring body, but also, for example, by interposing a suitable solid or fluid, gaseous or liquid media. In this way, the coupling can also take place, for example, in the emission of an acoustic pressure wave from the substance to the measuring body, and if necessary also via a path through a gaseous medium. By suitable choice of media between the substance and the measuring body, an impedance matching can be provided to achieve the best possible coupling into the measuring body.

The excitation beam is advantageously injected into the substance in an area that is either directly in contact with the measuring surface or is otherwise coupled to it. The excitation beam can also be injected into the substance directly next to an area of the measuring surface that is coupled with or in contact with the substance to be analysed. The excitation beam can be transmitted through the volume, through an opening or bored hole in the measuring body, or in particular also at least through some sections of the material of the measuring body, or else past an external boundary of the measuring body in the immediate vicinity of the measuring body. If an opening/bored hole is provided in the measuring body for the excitation beam, it can pass completely through the measuring body or be formed as a blind hole and in this case, in the area of the measuring surface the material of the measuring body or else a coating of another material, for example with a thickness of 0.05 mm to 0.5 mm, in particular a thickness of 0.1 mm to 0.3 mm, can remain in place.

Due to the influence of the thermal and/or pressure wave on the first optical waveguide structure, the refractive index in at least some sections of the first optical waveguide structure is changed and a phase shift of the detection light is caused, which leads to a measurable change in the light intensity at least in one section of the first optical waveguide structure.

For the detection of such phase shifts, interferometric methods and devices are available, for example.

The invention therefore also relates to the use of an interferometric measuring method or an interferometric measuring device for the quantitative measurement of the temperature increase in a material during the passage of a thermal and/or pressure wave.

The measuring body can be formed by a carrier body, on which the detection light source and the first optical waveguide structure can be attached or arranged. The detection light source can either be arranged directly in front of an injection point of the first optical waveguide structure or connected to it by means of an optical waveguide. The detection light source can also be integrated directly into the optical waveguide structure as an integrated semiconductor element, for example arranged on the same substrate as the optical waveguide structure. The optical waveguide can be implemented as a fibre-optic cable or else as an integrated optical waveguide. For example, the measuring device itself can also constitute or contain a substrate on which integrated optical waveguides can be arranged. The material of the measuring body can be made transparent or not transparent for the excitation light. The measuring surface can be defined as the outer boundary surface of the measuring body which can be coupled with or brought into contact with the substance to be analysed, wherein a thermal and/or pressure wave can be transported from the substance through the measuring surface to the measuring body.

In the design of the measuring body, it may be provided that the first optical waveguide structure is arranged in relation to the measuring surface in such a way that it is influenced by pressure or thermal waves caused by absorption of the excitation light when the measuring body is coupled/in contact with the substance in the area of the measuring surface.

For example, it can be provided that at least one section of a projection of the first optical waveguide structure in the direction of the surface normal of the measuring surface is superimposed with this measuring surface.

It may also be more generally provided that at least one section of the first optical waveguide structure can be reached by a wave in a straight direction from the measuring surface, in particular from the area of the measuring surface in which the excitation beam passes through it.

It is advantageous if at least one section of the optical waveguide structure, in particular an interferometric element, more particularly at least one arm of an interferometer of the optical waveguide structure, is located within an imaginary cone, the axis of which is perpendicular to the measuring surface, the tip of which is located at the point at which the excitation beam penetrates the measuring surface and which has an opening angle of not more than 90°, preferably not more than 60° and, in particular, not more than 20°. The opening angle is defined as twice the angle between the cone axis and an envelope line of the imaginary cone.

In addition, it may be provided that at least one section of the first optical waveguide structure is less than 2 mm, preferably less than 1 millimetre, more preferably less than 0.5 mm, away from the measuring surface.

The aim is to ensure that the first optical waveguide structure is arranged relative to the measuring surface in such a way that thermal and/or temperature waves, which are induced in the substance by absorption of the excitation light when the measuring body is coupled in contact with the substance in the area of the measuring surface, lead to a measurable phase shift of the detection light in at least one part of the first optical waveguide structure.

The measuring surface can be designed as a plane surface, but can also have a concave surface or partial surface, on which a placed body or object can be well centred or positioned. The measuring surface can then have the shape of, for example, a partially cylindrical channel or a dome shape, in particular a spherical dome shape, the radius of curvature being, for example, between 0.5 cm and 3 cm, in particular between 0.5 cm and 1.5 cm. If the measuring surface is not completely flat, either the surface normal in the centre of a concave recess of the measuring surface or the surface normal of a plane surface of a body placed on the measuring surface will be understood to be the surface normal of the measuring surface. The surface normal can also be understood to be the surface normal of a plane surface, which forms its continuation by bridging the concave recess of the measuring surface.

The measuring body can also be coated in the area of the measuring surface with a material that conducts a thermal and/or pressure wave in as loss-free a manner as possible. For example, this material can be gel-like or solid, and it can also be transparent to the excitation beam, or it can have an opening in an area where the excitation beam passes through the measuring surface. For example, the coating may be rather thin with a thickness less than 1 mm or less than 0.5 mm, or it may be rather thick with a thickness greater than 0.5 mm, in particular greater than 1 mm, and more particularly greater than 2 mm.

The curved surface shapes mentioned above may be formed by a substrate of the measuring body, a uniformly thick coating being provided, or the substrate may have a plane surface, wherein a curved surface may be realized by the thickness profile of the coating.

The first optical waveguide structure may be arranged on the opposite side of the measuring body to the measuring surface or on the surface of the side of the measuring body facing the measuring surface. In this case, the measuring body can form a substrate, on the side of which opposite the measuring surface or directly under the measuring surface optical waveguides are mounted, for example by means of epitaxial vapour deposition technology.

The first optical waveguide structure can also be arranged inside the measuring body or substrate and surrounded on all sides by the material of the measuring body/substrate, in order to ensure, for example, a good supply and good dissipation of thermal or pressure waves. In this case, the first optical waveguide structure may be “buried” inside a substrate by means of a known manufacturing process, i.e. it is covered on all sides by a different type of material, which in particular has a different refractive index than an optical waveguide of the first optical waveguide structure itself. If the optical waveguide itself is formed by silicon, then it can be covered by silicon oxide, for example. The substrate/measuring body can also be made entirely or partially of silicon. The integrated optical waveguide can also be constructed of plastic, for example polyethylene or an optically transparent crystalline material. For example, the first optical waveguide structure can be arranged parallel to the measuring surface and/or in a plane parallel to the measuring surface. In general, the optically integrated optical waveguides can be designed, for example, as so-called strip or slot waveguides, which means as material strips in which light waves are guided, or as appropriately formed gaps or intermediate spaces (slots) between totally reflecting boundaries consisting of a defined boundary material.

In a device of the described type a modulation device to modulate the intensity of the excitation beam may be provided.

In this case, the intensity of the excitation beam can be controlled by mechanical blocking (mechanical chopper) as well as using a controllable shutter or deflection mirror device, or a body/layer with a controllable transmission. In addition, modulation can also be achieved directly by controlling the excitation light source/laser light source, or by a shutter or an electronic intensity control, which entirely or partially blocks or deflects the excitation beam on its way from the excitation light source/laser device to the substance to be analysed. This can also be carried out by an interferometric device or an electronically controllable piezo-crystal or liquid crystal, or by another electronically controllable device that changes the transparency or reflectivity for the excitation light beam. Such a device can be provided as an integral part of the laser installation or as a functional element that is functionally integrated into the measuring body/substrate. This is possible because three-dimensional functional structures of the integrated optics and electronics can be formed by means of single or multi-layered structuring of the substrate. MEMS structures (micro-electromechanical structures) can also be integrated into the substrate in this way, for example in order to create a controllable deflection mirror for light modulation.

One possible aspect of the method presented here is the focusing of the measurement of the response signal on selected depth ranges below the (spacing intervals from) substance surface. The parameter d has the greatest influence on the depth range measured using the method. It is defined as d=√(D/(π*f)), where D is the thermal diffusivity of the sample (e.g. here, skin) and f is the modulation frequency of the excitation beam. For further details on the thermal diffusivity of skin, reference is made to the following publications:
U. Werner, K. Giese, B. Sennhenn, K. Plamann, and K. Kölmel, “Measurement of the thermal diffusivity of human epidermis by studying thermal wave propagation,” Phys. Med. Biol. 37(1), 21-35 (1992).
A. M. Stoll, Heat Transfer in Biotechnology, Vol 4 of Advances in Heat Transfer, J. P. Hartnett and T. Irvin, eds. (New York, Academic, 1967), p 117.

It should be noted that in this disclosure, the same term “response signal” is used in several ways. On the one hand, it can describe the physical response to the excitation by the excitation beam, i.e. such as a sound wave, a temperature rise, or the like. On the other hand, it can also describe an optical or electrical signal that represents this physical response, i.e. the intensity of the detection light (as an example of an optical signal), or a measured value of the intensity, which is an electrical signal. For the sake of simplicity and coherence of the presentation, the same term “response signal” is used throughout, and it is clear from the context without further explanation whether it refers to the physical response (for example, a pressure wave or temperature wave), a physical consequence of that physical response (for example, a phase shift of the detection light), or the associated measurement signal (for example, the intensity of the detection light measured by a photosensor).

In order to eliminate response signals from the topmost layers of the substance for the purpose of improving the quality of the measurement, in one embodiment changes in the measurement values compared to previous measurements can be used if the measurements in the topmost layers change to a lesser extent or more slowly compared to other, deeper layers. This can be the case in an embodiment in measurements on human skin, where the topmost layers of the skin are in practice not subject to an exchange with the lower layers and therefore physiological parameters do not vary very much. The temporal derivative of measured values can also be used for response signals to exclude the signals from the topmost skin layers. In this way, the measurement or at least the evaluation can be limited to or focused on the interstitial fluid in the skin.

For this purpose, a measurement can comprise the acquisition of response signals for spectra that are acquired multiple times with different modulation frequencies of the excitation light source, combining the results for different modulation frequencies, for example by differentiating or forming the quotient of the measurement values of response signals for the same wavelengths and different modulation sequences. To perform such a measurement an apparatus with an appropriate control device for the excitation beam and an evaluation device for the spectra of response signals should also be provided.

A measuring device may also be provided for the direct or indirect detection of the light intensity in the first optical waveguide structure, in particular in a section in which the light intensity depends on a phase shift of the detection light in at least one part of the first optical waveguide structure due to a change in temperature or pressure. The measuring device can itself measure a light intensity in the first optical waveguide structure or the intensity of a detection light component decoupled at a coupling point. The measuring device can comprise a light-sensitive semiconductor element integrated into the substrate, such as a photodiode. This allows a light intensity to be measured directly. Indirect measuring methods can be provided, for example, by measuring other parameters such as the temperature or a field strength at the first optical waveguide structure.

It can be further provided that the detection device comprises an interferometric device, in particular an interferometer and/or an optical waveguide resonance element, in particular a resonance ring or a resonance plate.

An interferometer, in particular a Mach-Zehnder interferometer, can be provided as the interferometric device in which the detection light is divided by a beam splitter into two partial beams, which are routed via two separate arms of the interferometer. The two arms of the interferometer are exposed to the effect of the temperature and/or pressure wave to different degrees, with the measuring arm being more strongly exposed to the effect of the temperature and/or pressure wave than the reference arm, or the influence of the temperature and/or pressure wave with respect to a change in the refractive index being stronger in the measuring arm than in the reference arm. In the best case, the reference arm is completely unaffected by the effect of the temperature and/or pressure wave, while the measuring arm is fully exposed to the effect.

In addition to a direct measurement of a light intensity in the variants described above, the intensity of the detection light can also be measured indirectly by means of another parameter, such as a temperature or field strength, provided that the parameter to be measured depends on the light intensity.

In order to ensure that the measuring arm is more strongly exposed to the effect of the temperature and/or pressure wave, it may be provided, for example, that at least one section of a projection of the measuring arm of the first optical waveguide structure in the direction of the surface normal of the measuring surface is superimposed with this measuring surface.

In addition, for a high efficiency of the measurement it may be provided that at least one section of the measuring arm of the first optical waveguide structure is less than 2 mm, preferably less than 1 millimetre, more preferably less than 0.5 mm, away from the measuring surface. The reference arm may be further away from the measuring surface than the measuring arm, as described in more detail elsewhere in this application.

The aim is to ensure that the measuring arm of the first optical waveguide structure is arranged relative to the measuring surface in such a way that thermal and/or temperature waves, which are induced in the substance by absorption of the excitation light when the measuring body is in contact with the substance in the area of the measuring surface, lead to a measurable phase shift of the detection light in at least one part of the measuring arm of the first optical waveguide structure. The measuring arm and/or the reference arm of an interferometer can be advantageously oriented parallel to the measuring surface and/or run in a plane parallel to the measuring surface.

The light from both arms is recombined after passing through the arms and, depending on the phase shift of the detection light in the arm that is more strongly exposed to the effect, the two mutually phase-shifted partial beams of the detection light at least partially cancel each other out. The measured light intensity is then minimized, unless the phase shift exceeds 180 degrees and in the extreme case passes through multiple full cycles of 360 degrees (2 Pi) each. In this case, in the course of the development of the temperature and/or pressure increase, the zero crossings can also be counted in the phase cancellation of the two partial beams in order to determine an absolute phase shift. In many cases, however, due to the small temperature and/or pressure effects to be detected, the phase shift will not exceed 180 degrees. The operating point of the interferometer can then be set in such a way that the resulting changes in light intensity are monotonically mapped onto the pressure/temperature changes.

The following measures may be taken to ensure that the measuring arm is more exposed to the effect of the temperature and/or pressure wave than the reference arm, or that the influence of the temperature and/or pressure wave with respect to a change in the refractive index is stronger in the measuring arm than in the reference arm:

The measuring arm, with or without a ring resonator integrated into or connected to it, is in mechanical contact with the substrate. The optical waveguides of the measuring arm can be connected to the substrate in a positive-fitting and/or materially-bonded and/or force-fitting manner. It can also be pressed against the substrate or clamped to it.

If the interferometric device contains only one or more ring resonators or other optical waveguide resonance elements, these may also be in mechanical contact with the substrate. The optical waveguide(s) of the ring resonator(s) or resonance elements can also be connected to the substrate in a positive-fitting and/or materially-bonded and/or force-fitting manner. They can also be pressed against the substrate or clamped to it.

An interferometer or one or both of its measuring arms, in the same way as one or more ring resonators or other resonance elements, can be integrated into the substrate and, for example, be manufactured together with the substrate in an integrated manufacturing process.

A reduced effect of the temperature and/or pressure wave on the reference arm or a reduced effect on the refractive index of the optical waveguide(s) or the optical light path in the reference arm can be realized, inter alia, by at least one part of the optical waveguide or even the entire optical waveguide of the reference arm being formed of a fibre-optic cable, in which case the fibre-optic cable, in some sections or over the majority of its length or entirely, is arranged outside the substrate, in particular spaced apart from it. The fibre-optic optical waveguide can also run outside the material of the substrate, for example in a recess of the substrate, without being connected to the material of the substrate.

At least a part of the reference arm can also extend separately from the measuring arm through a second substrate, on a second substrate, or in or on a substrate part that is separated or shielded or spaced apart from the substrate, at least in sections.

In this case, the reference arm or the second substrate or the partial substrate may be separated from the substrate, at least in sections, by an air gap or barrier. Possible substances for the barrier can be those that are softer or less stiff than the substrate material and consist, for example, of a plastic, an elastomer, an organic material, a textile, paper or a foam.

In any case, for example, at least 10%, in particular at least 20%, more particularly at least 30% of the optical length of the reference arm may be located in an area of the same substrate as the measuring arm or of another substrate, which is at least 2 mm, in particular at least 5 mm, more particularly at least 8 mm, apart from the measuring arm. This area of the reference arm may be further away from the measuring surface than the measuring arm. The said portion of the refraction arm may be advantageously located in an area that is not reached by the thermal and/or pressure wave, or at least less influenced than the area in which the measuring arm is located.

If the measuring arm and the reference arm are at least partly made up of different materials, a beam splitter may be provided for splitting the detection light over the measuring arm and the reference arm. This beam splitter can be integrated into the substrate or provided separately from it. The beam splitter can be designed to distribute the detection light over an integrated optical waveguide and a fibre-optic cable, or over two integrated optical waveguides or two fibre-optic cables.

Regardless of the arrangement and distance of the measuring arm and the reference arm from each other, the measuring arm and the reference arm may be at least partially or completely made of different materials, the material of the reference arm being selected such that its refractive index is influenced by the effect of the thermal and/or pressure wave to a lesser extent than the refractive index of the material of the measuring arm. This can be achieved, for example, by selecting different raw materials for the measuring arm and reference arm, or by different doping of the same raw material in the measuring and reference arms. It may also be provided that on at least a portion of the length of the reference arm the detection light is passed through a fluid, in particular a gas, for example air or nitrogen, or a transparent liquid.

If a ring resonator or other optical waveguide resonance element is used as a detection device, this may be arranged in a plane that is parallel to the measuring surface. This means that all sections of the ring resonator are exposed as evenly as possible to the effect of a temperature and/or pressure wave from the measuring surface incident on the ring resonator. If a ring resonator or another resonance element is used as a detection device, either exclusively or combined with an interferometer, then instead of a single ring resonator or resonance element a plurality of optically cascaded or parallel connected ring resonators or resonance elements may be used to shape the frequency response as required. The operating point(s) can be adjusted by temperature control or by adjusting a mechanical pressure on the ring resonators/resonance elements. The operating point can be set in such a way that a maximum temperature or pressure sensitivity or a maximum measuring range with a monotonic dependence between temperature or pressure and the light intensity in the resonance ring/resonance element is produced.

The device for analysing a substance may include an evaluation device which determines the change in intensity of the detection light detected by the detection device, and from this an absorption strength as a function of the wavelengths of the excitation beam. Due to the modulation of the excitation beam, the detection light intensity can be measured with and without the influence of the thermal and/or pressure wave to be measured and their difference or ratio or other relationship variable between these values can be evaluated.

In particular if an optical waveguide resonance element is provided as the interferometric element, or an interferometer with two measuring arms with measuring sections that are arranged and oriented relative to the measuring surface such that they are reached consecutively by the thermal and/or pressure wave, the evaluation device may also be configured in such a way that the course or temporal profile of the intensity of the detection light, i.e. the course of the de-tuning of the resonance element by changing the refractive index, or the course of the phase shift in the two measuring sections/measuring arms of the interferometer, is recorded while a thermal and/or pressure wave or one or more wavefronts passes through it.

Particularly when an interferometer with multiple measuring sections is used, if both of these are exposed to the thermal and/or pressure wave, the phase shifts can be compensated so that no change in the detection light intensity can be observed. However, if the measuring arms/measuring sections are positioned/oriented such that they are reached by the wave consecutively, then an intensity course or temporal profile of the detection light will arise that reflects the different and time-shifted effect of the wave on the different measuring sections and thus allow an evaluation, since there will be time segments in which the wave has a different effect on the different measuring sections.

In the case of the optical waveguide resonance elements, a temporal profile of the intensity of the detection light is obtained which reflects the amplitude of the passing wave.

In the case of a modulated excitation beam and the resulting thermal and/or pressure waves that pass through, a suitable parameter, for example the amplitude, of the periodic change in the intensity of the detection light, can be used for evaluation.

It may also be provided that the optical waveguide structure, in particular the interferometric device of the first optical waveguide structure, comprises at least one fibre-optic cable, which is fixedly connected to the measuring body at least in some sections.

A fibre-optic cable is available at low cost and due to its flexibility can be easily adapted to the existing requirements. However, it must be brought into contact with the measuring body in order to be affected by the pressure and/or thermal wave. For this purpose, the optical waveguide can be adhesively bonded to the substrate/measuring body or connected to it in a form-fitting or force-fitting manner. For example, the fibre-optic cable can be mounted in a clamping device of the substrate.

It may also be provided that an optical waveguide of the first optical waveguide structure, in particular an interferometric device of the first optical waveguide structure, is integrated in a substrate of the measuring body or is connected to a substrate, the first optical waveguide structure having in particular at least one silicon optical waveguide, which is connected to an insulating substrate or is integrated into an insulating substrate, and in particular the silicon optical waveguide also being at least partially covered by an insulator, in particular a silicon oxide, for example SiO₂.

In this case, the first optical waveguide structure can be constructed on the substrate using the known means from integrated optics, in which areas of different refractive index can be created, for example by selective doping of the substrate material or by the formation of oxide layers or other layers from reaction products. Such integrated optical waveguide structures can be provided in or on a silicon wafer. An optical waveguide structure can also be formed in a polymer body. In addition, integrated optical waveguide can be formed, for example, using material combinations GeO₂—SiO₂/SiO₂, GaAsInP/InP.Ti:LiNbO₃.

In addition, it may be provided that the excitation beam passes through the material of the measuring body, in particular in the area of the measuring surface of the measuring body or an area neighbouring the measuring surface or an area immediately adjacent to the measuring surface, wherein the measuring body or the area penetrated by the excitation beam is transparent to the excitation beam.

The transparency of the measuring body and, in particular, also of a coating of the measuring body, will be provided in the wavelength range of the excitation beam or excitation light beam. The transparency may also not be provided completely, so that a certain absorption of the excitation beam will have to be allowed for. The layer of the measuring body which is penetrated by the excitation beam can then be designed as thin as possible, for example thinner than 1 mm, for example only as a thin layer in the area of the measuring surface.

It may also be provided that the excitation beam is guided within the measuring body or on the measuring body by a second optical waveguide structure. The second optical waveguide structure is then designed in such a way that it can guide light or radiation in the wavelength range of the excitation beam in as lossless a manner as possible. The excitation beam is coupled into an optical waveguide of the second optical waveguide structure and decoupled from it in the area of the measuring surface and directed toward the substance to be examined. A beam-shaping optical element, in particular a focusing or collimating element, may be provided at the injection point and/or at the decoupling point, which may be provided separately from or integrated into the optical waveguide structure. The first and the second optical waveguide structures can be provided separately and separated and spaced apart from each other. Due to the linearity of the wave equation however, they can also intersect each other without any interaction, so that there are regions in the optical waveguide structures that are passed through by both the excitation beam and the detection light. In the extreme case, the first and the second optical waveguide structure can be identical and have the necessary injection and decoupling points for the excitation light beam as well as for the detection light. It is also possible that the reaction of the pressure and/or temperature change in the optical waveguide structure on the excitation light is detected and taken into account during the evaluation. The laser device for generating the excitation beam may be integrated into the measuring body and at least one or more or all of the electronic elements of the laser device can be provided on a substrate of the measuring body, in particular on the same substrate that also supports the integrated optical elements. Electrical elements of the laser device and integrated optical elements can be produced or arranged on one or more connected substrates in a joint production process and/or in a series of successive production steps. This results in an extremely space-saving arrangement. This integrated arrangement can be provided both when a second optical waveguide structure is provided for guiding the excitation beam, and in the case that the excitation beam is directed toward the substance to be analysed through an opening in the measuring body.

It may also be provided that the excitation beam between the laser device and the substance to be analysed passes through a continuous opening of the measuring body, wherein the opening ends in particular at a distance in front of the measuring surface or penetrates the measuring surface or is arranged in a region which is directly adjacent to the measuring surface and/or adjoins it.

In this case, the excitation beam propagates in the opening and in some cases emerges from the opening on the side of the measuring body facing the substance to be analysed without having passed through the material of the measuring body. A thin layer of the measuring body can also remain in place in the area of the measuring surface, so that the opening does not completely pass through and ends at a distance in front of the measuring surface. It is important that the volume of the substance into which the excitation beam is irradiated is adjacent to the measuring surface and is in contact with it or is coupled with it in another suitable way, so that the generated temperature and/or thermal wave at least partially strikes the measuring surface and is directed through it into the measuring body or to the detection device.

The continuous or largely continuous opening in the measuring body can form a straight channel, but it can also form a channel with curves or bends, wherein the excitation beam can then be guided through the channel by means of deflecting or reflecting elements. The opening can continue through a coating of the measuring body, but it can also end at the coating, so that the excitation beam is guided through the coating.

If the excitation beam passes through at least one specific layer of the measuring body, then if the material of the measuring body at least partially absorbs the excitation beam, the excitation beam can already generate a temperature increase of the measuring body, but one which is precisely calculable. The periodic operation of the excitation light source results in thermal waves in the measuring body, which in some cases reach the detection device and can be detected thereby. This effect can be calculated and subtracted from the useful signal.

It may also be provided that the excitation beam is steered directly along an external boundary of the measuring body onto the substance to be analysed and penetrates into the substance in the extension of the measuring surface next to the measuring body. The first optical waveguide structure of the detection device, for example an interferometer, can then be provided in the measuring body directly next to the area in which the excitation beam passes through the imaginary continuation of the measuring surface, so that the thermal and/or pressure wave emerging from the substance there at least partially enters the measuring body and reaches the first optical waveguide structure.

In a further embodiment it can be provided that the measuring body is formed as a flat body, in particular as a plane-parallel body in the form of a plate, wherein in particular the thickness of the measuring body in a direction perpendicular to the measuring surface is less than 50% of the smallest extension of the measuring body in a direction extending in the measuring surface, in particular less than 25%, more particularly less than 10%.

Such a design can result from the use of a flat substrate, such as a wafer, for the integrated optics. The required thickness of the measuring body is then limited by the space required for the detection device.

A further embodiment can provide that the measuring body comprises a mirror device for reflecting the excitation beam irradiated by the laser device onto the measuring surface or carries such a mirror device.

This is particularly important if the laser device is aligned in such a way that the excitation beam in the laser device is not generated perpendicular to the measuring surface, for example if the laser device is to be installed next to the measuring body in a space-saving manner or oriented at an angle to it.

It can also be provided that the excitation beam is oriented into the measuring body parallel to the measuring surface or at an angle of less than 30 degrees, in particular less than 20 degrees, more particularly less than 10 degrees or less than 5 degrees to the measuring surface, and that the excitation beam is diverted or deflected towards the measuring surface, wherein the excitation beam in particular passes through the measuring surface or an imaginary continuation of the measuring surface in the region of a continuous opening in the measuring body.

In this case, the laser device for generating the excitation beam may be arranged in a particularly space-saving manner and aligned in such a way that it generates or decouples the excitation beam parallel to the measuring surface or at one of the aforementioned shallow angles with respect to the measuring surface.

In addition, it may be provided that at least one heat sink in the form of a solid body or material is arranged in the measuring body behind and/or next to the detection device as seen from the measuring surface, in particular adjacent to and in thermal contact with it, wherein in particular the specific thermal capacity and/or specific thermal conductivity of the body or material of the heat sink is greater than the specific thermal capacity and/or specific thermal conductivity of the material of the detection device and/or the optical waveguide structure and/or the substrate of the optical waveguide structure and/or the other materials from which the measuring body is composed.

In principle, it may be advantageous to provide a heat sink in or on the measuring body in order to dissipate the heat that is introduced into the detection device by a thermal wave as quickly as possible, so that even at high modulation frequencies of the excitation beam a heat energy equilibrium or temperature equilibrium is created which allows the intermittently irradiated heat quantities or the temperature changes generated thereby to be measured without being distorted by temperature changes in the past.

In some applications, in particular in interferometric applications, more particularly in a Mach-Zehnder interferometer, it is also advantageous to expose one measuring arm to the temperature changes or pressure waves and to shield the other arm, the reference arm, from the temperature changes or pressure waves. For this purpose, it may also be useful to space the reference arm apart from the measuring arm and/or provide a barrier that at least partially shields a part of the detection device, in particular the reference arm of an interferometer, from the effect of the thermal and/or pressure wave. Such a barrier may consist, for example, of a material that has a lower thermal conductivity than the material of the measuring body or of a substrate of the measuring body. The material can also be more flexible or elastic or more easily deformable than the material of the measuring body or of a substrate of the measuring body to provide mechanical decoupling of a pressure wave. A barrier can also be formed by a gas gap, which can be introduced into a substrate, for example by etching or a material-removing process or else by an additive manufacturing process.

To space the reference arm of a Mach-Zehnder interferometer apart from the measuring arm, it may also be provided that the measuring arm and reference arm are arranged in different planes of the substrate, wherein the plane in which the reference arm is located is a greater distance away from the measuring surface than the plane in which the measuring arm is arranged.

A temperature and/or pressure change can also be detected by means of an optical waveguide resonance ring in which the detection light propagates in resonance under suitable conditions. If the temperature and/or pressure conditions change, the resonance is detuned by a change of the refractive index and a partial or complete cancellation takes place. Such a resonance ring has a sensitivity that is ideally much higher than even that of a Mach-Zehnder interferometer. Such a resonance ring can also be integrated into one arm, preferably the measuring arm, of a Mach-Zehnder interferometer.

It may also be provided in an embodiment of the invention that the optical waveguide structure of the detection device comprises at least two measuring sections, arranged in particular on different arms of an interferometer and in which the refractive index changes as a function of pressure and/or temperature changes, in particular of a pressure and/or thermal wave, so that a phase shift occurs in the detection light passing through the measuring sections followed by a resulting intensity change in the detection light in a further section as a function of pressure and/or temperature changes, the two measuring sections being arranged in the measuring body in such a way that they are passed through by a pressure and/or thermal wave which propagates through the measuring body starting from the measuring surface, in particular from the region of the measuring surface in which the excitation beam penetrates it, sequentially, in particular in time intervals temporally shifted relative to one another or with a time delay.

A pressure and/or thermal wave that propagates through the measuring body starting from the area of the measuring surface in which the excitation beam passes through the latter, initially reaches a first of the measuring sections and temporarily changes the refractive index there during its transit. In the time interval during which this modified refractive index is active, a first phase shift relative to the detection light is generated, which passes through the second measuring section (the detection light passes through both measuring sections in parallel). This phase shift can be detected by the intensity measurement of the detection light, described above. The wave then reaches the second measuring section and manifests its effect there, by also changing the refractive index there for a time interval. If the two time intervals overlap, the phase shifts are at least partially neutralized for the duration of the overlap. After that, if the phase shift takes place only in the second measuring section, the effect of the intensity change of the detection light occurs again. This temporal profile can be recorded by an evaluation device and from this, the change in the refractive index in the first measuring section and the second measuring section can be determined. The determined change in the refractive index can be attributed to a change in temperature and/or pressure, which is a measure of the absorption strength of the excitation beam in the substance to be analysed. In this case, the aforementioned measuring sections with their optical waveguide longitudinal axes advantageously run transversely, in particular at right angles, to the propagation direction of the pressure and/or temperature wave in the measuring body and more particularly, when seen from the area of the measuring surface at which the excitation beam passes through it, one behind the other.

An evaluation device is also advantageously provided which uses the intensity change of the detection light in the optical waveguide structure to determine a magnitude of the phase shift change of the detection light in a measuring section and from this, the change in the refractive index. From this change in the refractive index, the pressure and/or temperature change in the measuring sections can be determined and from this, the absorption strength of the excitation beam in the substance to be analysed.

The invention also relates to a sensor which can be used, for example, for a device the type described above, with a measuring body which has a measuring surface and which is to be at least partially coupled, in particular brought into contact, with a substance in the area of the measuring surface for measuring a temperature and/or pressure wave,

and having a detection device, which is at least partially integrated into the measuring body or connected to it, comprising the following:
a source for coherent detection light, and
a first optical waveguide structure, which can be connected or is connected to the source for the detection light and which guides the detection light, the refractive index of which at least in sections is dependent on the temperature and/or pressure,
at least one section in which the light intensity depends on a phase shift of the detection light in at least one part of the first optical waveguide structure due to a change in temperature or pressure, the first optical waveguide structure comprising an interferometric device, in particular an interferometer and/or an optical waveguide resonance ring or other optical waveguide resonance element, and
a measuring device for detecting the light intensity in or at the interferometric device.

All of the features explained in this application for the design of the temperature sensor of the analysis device according to the invention may also be used to implement a sensor independently of the analysis device for other purposes, in particular all the described arrangements, designs, material choices, production types, and shapes of an integrated optical resonance ring or the measuring arm and the reference arm of an interferometer.

With this sensor, temperature changes or pressure waves can be measured, which can be detected by means of refractive index changes. In addition to the purposes explained above, the sensor can therefore also be used for vibration measurements, e.g. seismic measurements or mechanical impulse measurements. Due to its short response time, the sensor is thus qualified for measurements in which other sensors such as MEMS sensors cannot be used due to their inertia.

In addition to a device of the type explained above, the invention also relates to a method for operating such a device, wherein it is provided that a modulated excitation beam is directed, in particular through the measuring body, toward the substance to be analysed and that a temporal light intensity characteristic or a periodic light intensity change is detected by the detection device, these being detected for a plurality of wavelengths of the excitation beam by measuring the light intensity change in the first optical waveguide structure or by measuring the light intensity of light decoupled from the first optical waveguide and obtaining an absorption spectrum of the substance to be analysed from the acquired data.

In such a method, it may also be provided that the measurement is carried out for different modulation frequencies of the excitation beam and that a corrected absorption spectrum is determined from the combination of absorption spectra obtained. This allows a depth profiling of the concentration of an analysed substance in the substance under examination to be determined, or interfering effects from certain depth ranges can be reduced or eliminated by means of mathematical combination or correlation.

Generally, the invention also comprises a method for analysing a substance, in particular using a device of the type explained above, wherein in the method
with an excitation transmission device, at least one intensity-modulated electromagnetic excitation beam with at least one excitation wavelength is generated, the excitation transmission device irradiates the at least one electromagnetic excitation beam into a volume of substance which is located below the surface of the substance,
a response signal in the form of a light intensity in the first optical waveguide structure is detected with a detection device, and
the substance is analysed on the basis of the detected response signal, wherein response signals, in particular temporal response signal waveforms for different wavelengths of the excitation beam are determined and from the decay behaviour of the response signals after the end of each modulation phase in which the excitation beam has a high intensity, information about the depth profile under the surface of the substance to be analysed is obtained, in which the excitation beam is absorbed and the thermal and/or pressure wave is generated.

It may also be provided that
using different modulation frequencies of the excitation transmission device a plurality of response signal wave forms are determined and
a plurality of response signal waveforms at different modulation frequencies are combined with one another and wherein
information specific to a depth range under the surface of the substance is obtained from these.

In particular in the case of a pressure change being detected, the detection device can also be used to detect a response signal in the form of a sound wave which is generated in the substance to be analysed by the absorption of the excitation beam and which travels to the measuring surface and to the detection region at a known speed (in human tissue, approx. 1500 m/s). By means of an evaluation device connected to a modulation device for the excitation beam, due to the good temporal resolution of the measurement of the response signals a phase shift between the modulation of the excitation beam and the response signal can be measured, and thus the depth in the tissue in which the absorption took place can be determined. Since the signals are often a superposition of different response signals from different tissue layers, the signals can be interpreted by building a model with a plurality of absorption sites distributed at different depths of the substance and their associated absorption strengths, as well as transit times to the substance surface, wherein the absorption strengths are then fitted to the temporal response signal waveform so that the response signal waveform can be reconstructed. From this, the absorption strengths and thus the local concentrations of the component to be detected in the substance can be determined.

Alternatively or additionally, different measurements can also be carried out at different modulation frequencies and the response signals at different modulation frequencies can be combined, in particular to cancel out and eliminate signals from upper tissue layers, as these are particularly susceptible to errors due to contamination by dirt and dead skin cells.

The above-mentioned device can also be advantageously combined
with at least one other detection device that is arranged adjacent to and/or directly adjoining the measuring surface, the other detection device having a contact device with at least two electrodes for detecting piezoelectric signals, said electrodes being located opposite each other on different sides of a detection region. In the detection region, a material is arranged that changes its electrical resistance or generates an electrical signal as a function of temperature and/or pressure changes, in particular due to a piezoelectric effect.

This additional detection device can be used, for example, to measure a temperature or a pressure in an alternative way, wherein this measurement can be used as a reference measurement for an ambient temperature or an ambient pressure or also for measuring the thermal and/or temperature wave emitted from the substance to be analysed, in order to correlate the measurements obtained by the detection device with measurements from the other detection device.

In the following the invention will be illustrated and explained in further detail based on figures of a drawing.

They show:

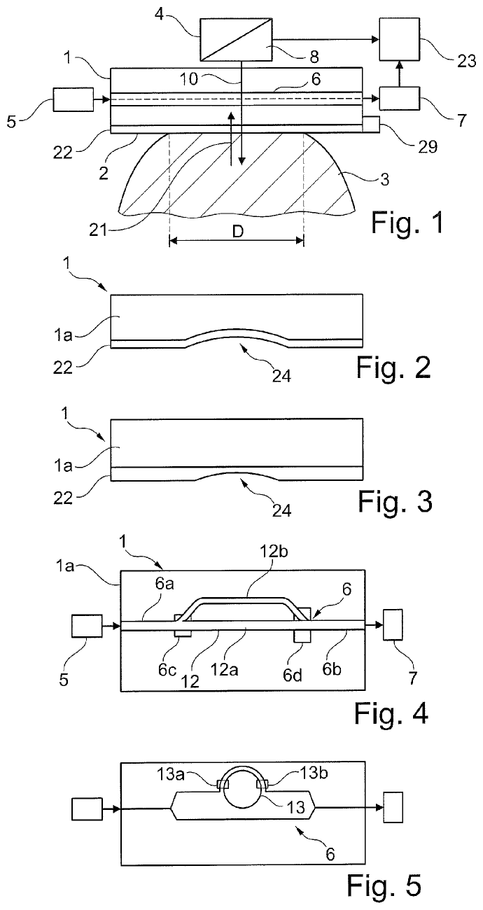

FIG. 1 a schematic side view of a measuring body with a laser device and a detection device,

FIG. 2 a side view of a measuring body,

FIG. 3 a side view of a further measuring body,

FIG. 4 a plan view of a first optical waveguide structure on a measuring body;

FIG. 5 a plan view of another implementation of a first optical waveguide structure on a measuring body,

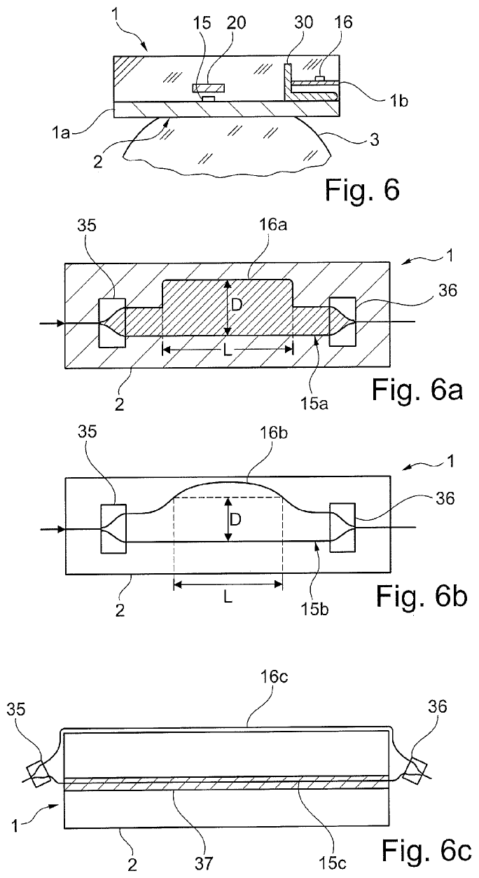

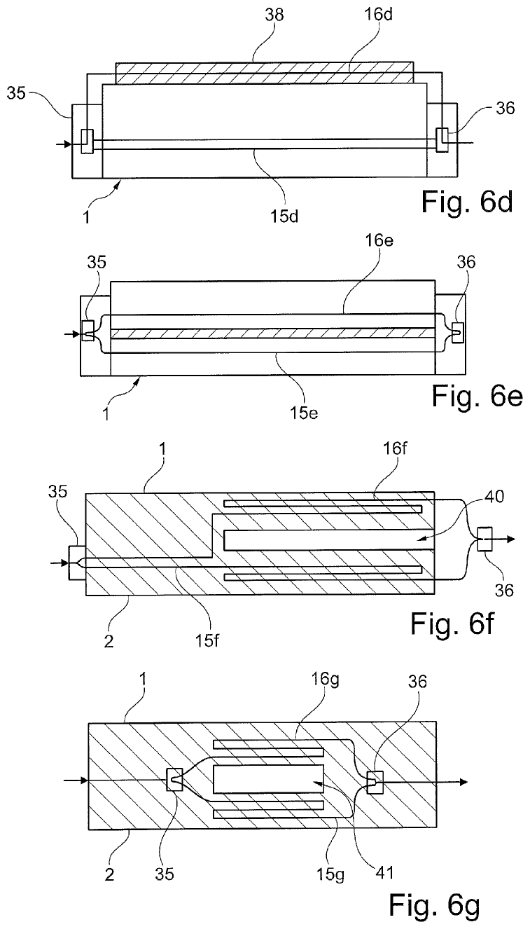

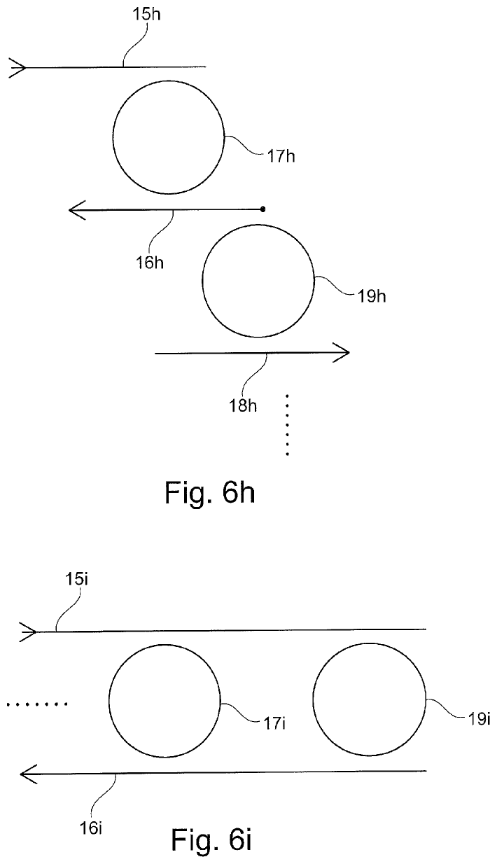

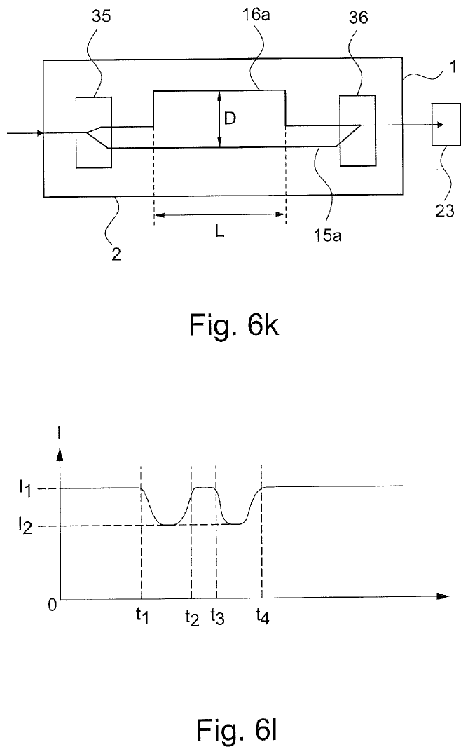

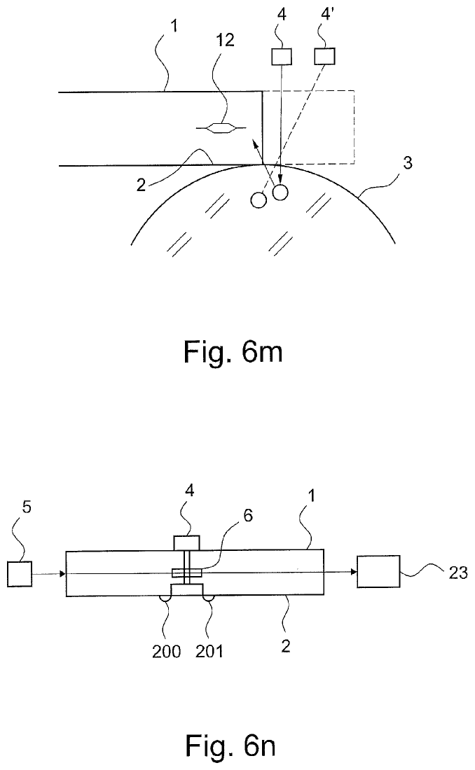

FIG. 6 a cross section through a substrate with integrated optical waveguides,

FIGS. 6a-6i different embodiments of one or more substrates with an interferometric device, wherein the hatching of the measuring body is shown in some illustrations and omitted in others for the sake of clarity,

FIG. 6k an embodiment with an interferometric device, in which the temporal profile of the phase shift/refractive index change can be measured as a function of the passage through the different measuring sections by a pressure and/or thermal wave,

FIG. 6l the temporal waveform or profile of the phase shift of the detection light in the measuring sections during the passage of a pressure and/or thermal wave,

FIG. 6m a path of an excitation beam past an outer boundary surface of a measuring body into the substance, as well as the position of an interferometric device,

FIG. 6n a measuring body with an acoustic coupling element for coupling to the substance to be analysed,

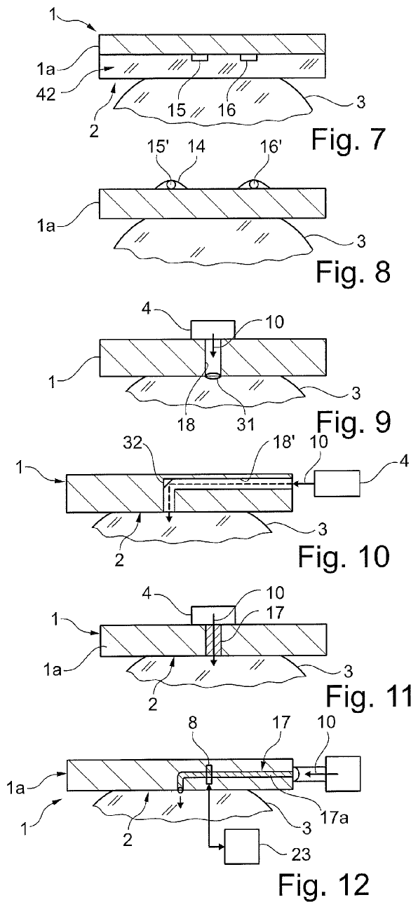

FIG. 7 a cross-sectional view through another substrate with integrated optical waveguides,

FIG. 8 a cross-section through a substrate with optical waveguides glued onto it,

FIG. 9 a cross-sectional view of a substrate with a continuous opening for an excitation beam,

FIG. 10 a cross-sectional view of a substrate with a further continuous opening for an excitation beam,

FIG. 11 a cross-sectional view of a substrate with a second optical waveguide structure for an excitation beam,

FIG. 12 a cross-sectional view of a substrate with a further implementation of a second optical waveguide structure for an excitation beam,

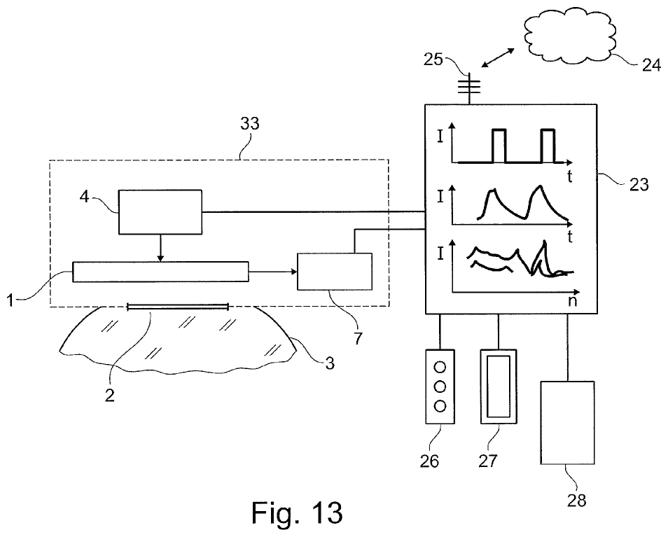

FIG. 13 a schematic overview of the device for analysing a substance with a processing device for measuring results and output devices for signals,

FIG. 14 to 16 an arrangement with a substrate, to which the excitation light source and the detection light source as well as a detector are connected, and in which another substrate with integrated optical elements can be inserted,

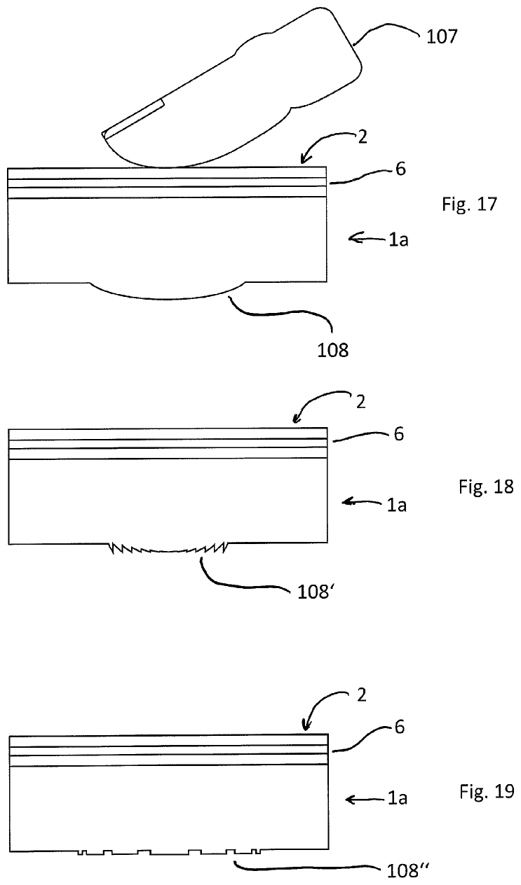

FIG. 17 a cross-section of a measuring body with a first integrated lens and with a finger placed on the measuring surface,

FIG. 18 a cross-section of a measuring body with a second integrated lens,

FIG. 19 a cross-section of a measuring body with a third integrated lens,

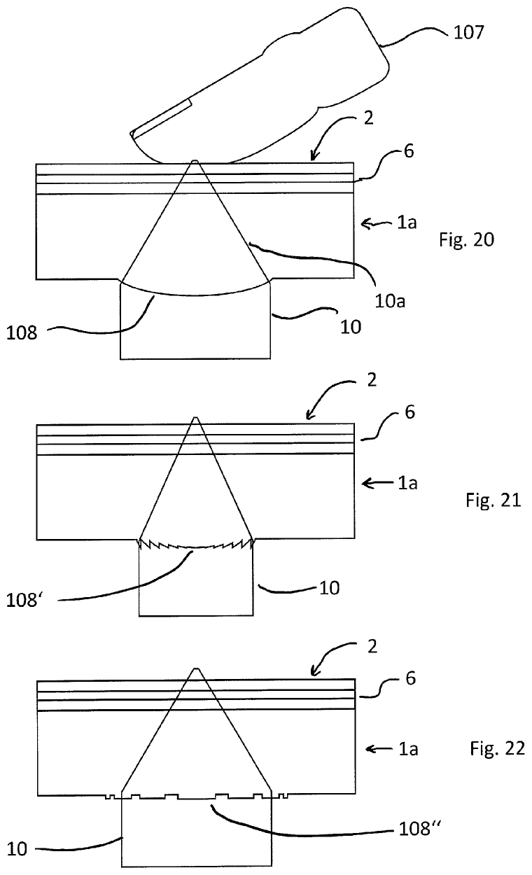

FIG. 20 a cross-section of a measuring body with a first integrated lens and an excitation beam,

FIG. 21 a cross-section of a measuring body with a second integrated lens and an excitation beam,

FIG. 22 a cross-section of a measuring body with a third integrated lens and an excitation beam, and

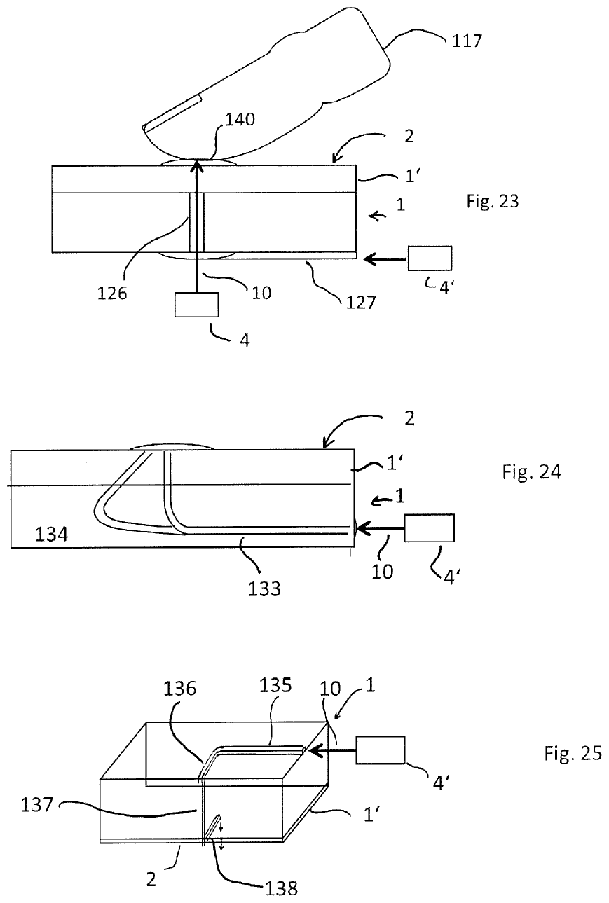

FIGS. 23, 24, 25 several arrangements with a measuring body and an excitation light source in the form of a laser light source or excitation light source, in particular a laser device, wherein the excitation light beam is guided to the measuring surface by the measuring device by means of an optical waveguide integrated into a substrate of the measuring body.

FIG. 1 shows a cross-sectional view of a measuring body 1, the internal structure of which is not discussed in detail in this figure. Within the measuring body 1, a first optical waveguide structure 6 is shown schematically, into which coherent detection light is irradiated by a detection light source 5. A measuring device 7 is used to detect a light intensity in the first optical waveguide structure 6, which is dependent on the pressure or temperature acting on the optical waveguide structure 6.

The detection light source 5 can be designed as a laser or laser diode and be arranged on or fixed to the measuring body 1. The detection light source 5 can also be flexibly connected to the first optical waveguide structure 6 by means of a fibre-optic cable. In addition, the detection light source 5 can be integrated into a substrate (not shown here) within the measuring body 1 as a semiconductor element and connected there to a first optical waveguide structure.

The measuring device 7 can also be connected to the first optical waveguide structure 6 directly by means of a coupler, or connected to it by means of an integrated optical waveguide or a flexible fibre-optic cable (not shown here). However, the measuring device 7 can also be integrated into the measuring body and be implemented on a substrate of the measuring body 1 as a semiconductor element. For example, the measuring device 7 can be designed as a light-sensitive semiconductor element, for example as a photodiode.

In addition to the above components, a temperature measuring device for measuring the absolute temperature of the measuring body 1 can be provided to take into account an average temperature measured over longer time intervals, for example one tenth of a second, half a second, one or more seconds, depending on the time constant of the other sensors in the evaluation of the measurements. This allows, for example, the temperature dependence of a photodiode or other semiconductor light sensor to be corrected. This can be useful, for example, for the evaluation of the light intensity measured by the measuring device 7, which can be improved by a temperature correction. Alternatively, a temperature stabilisation device 29 can be provided, which contains a heating or cooling element and maintains the measuring body 1 at a constant temperature. For example, this temperature can correspond to an average temperature that can be fixed, for example at 20° C., but it can also correspond to an average body temperature of a patient whose body tissue or bodily fluid is to be measured and which can thus be approximately 37° C. or 30° C. (exposed skin surface).

FIG. 1 shows a laser device 4, which can be implemented as a quantum cascade laser or a laser array. The quantum cascade laser can be designed in such a way that it is at least partly tuneable with respect to its wavelength, in particular in the infrared range, more particularly tuneable in the mid-infrared range. If the laser device 4 is set up as a laser array, individual laser elements of the array can be tuneable, adjustable or fixed at specific wavelengths. The wavelengths of the individual laser elements can be set, for example, in such a way that they correspond to the wavelengths of absorption maxima of a component to be detected in the substance to be analysed, i.e., the absorption maxima of glucose, for example. The wavelength of the excitation beams for the example of the blood sugar measurement described here can be preferably chosen in such a way that the excitation beams are significantly absorbed by glucose or blood sugar. The following glucose-relevant infrared wavelengths (vacuum wavelengths) are particularly suitable for measuring glucose or blood sugar and can be set individually or in groups simultaneously or in succession as fixed wavelengths for measuring the response signals: 8.1 μm, 8.3 μm, 8.5 μm, 8.8 μm, 9.2 μm, 9.4 μm and 9.7 μm. In addition, glucose-tolerant wavelengths that are not absorbed by glucose can be used to identify other substances present and exclude their influence on the measurement.

However, since the device can also be used, for example, to detect and analyse other biological or chemical substances, the absorption maxima of the substances to be detected are also applicable here. The number of transmission elements of a laser array can be a number from 10 to 20 or a number from 10 to 30 elements or even a number larger than 30 transmission elements.

The laser device 4, which can also be called an excitation beam generating device or excitation beam transmission device, has a modulation device 8 that generates a modulated laser beam. In this case, the modulation device 8 can be arranged, for example, in the controller of the laser device 4. For example, the modulation frequency can be between 100 Hz and a few megahertz, or even several hundred megahertz. The important point is that the first optical waveguide structure 6 has a suitable response time and can respond to also intensity-modulated pressure or thermal waves that are incident according to the modulation frequency. This is the case when using the interferometric detection devices described in further detail below.

Light from the laser device 4 is incident as excitation beam 10 through a measuring surface 2, which is shown as the lower surface of the measuring body 1, into the area labelled D and in which the substance 3 to be analysed comes into contact with the measuring surface 2. After absorption of the excitation light beam 10 in the substance 3, a temperature and/or pressure wave 21 is guided from the substance to the measuring body 1 and strikes the first optical waveguide structure 6. The temperature and/or pressure change causes an intensity change of the detection light there, which is detected by means of the measuring device 7 and passed on to a processing device 23. The processing device 23 can be equipped with a lock-in amplifier which amplifies the signals synchronously with the modulation of the excitation beam 10.

Optionally, the measuring body 1 can be provided with a coating 22 in the area of the measuring surface 2, to which the substance 3 to be analysed can be applied directly. This can be useful to protect a substrate material provided in the measuring body 1 or to promote the mechanical and/or thermal coupling of the substance 3 to the first optical waveguide structure 6. The material of the coating 22 should be designed in such a way that it transmits pressure and thermal waves well. It can also be chosen to be transparent to the excitation beam 10. A covering layer 22, which can also be provided in principle between the first optical waveguide structure 6 and a substance to be analysed, for example on a surface of the first optical waveguide structure 6, can also be used to prevent a direct interaction of radiation within the first optical waveguide structure 6, or at least the interaction of an evanescent part of this radiation outside the actual optical waveguide structure 6, with a substance applied to the measuring surface 2, since such a contact could have a retroactive effect on the radiation in the first optical waveguide structure 6.

An acoustic coupling of the measuring body to the substance to be analysed can also be provided, in which the measuring body absorbs the waves generated in the substance by means of a medium inserted between the measuring body and the substance. The medium can be a fluid, i.e. in gaseous or liquid form, so that a distance can be provided between the measuring body and the substance, for example in the form of a cavity or a recess in the measuring body. The opening of the cavity can then be placed on the substance, so that the wave can enter the measuring body through the cavity. The wall of the cavity, i.e. the outer surface of the measuring body, can be coated with a material that produces good acoustic coupling, i.e. impedance matching. Such an acoustic coupling is shown and explained in more detail below using FIG. 6n.

FIG. 2 shows in a side view that the measuring body 1 can form a trough 24, which is covered with the coating 22. The trough 24 is provided to allow the substance 3 to be analysed to be placed on the measuring surface 2 in this area. This provides orientation for the user of the device. In addition, the trough provides mechanical stabilisation when a part of the body, for example a finger pad, is placed on the measuring surface 2.

FIG. 3 shows as an alternative design that the trough 24 is formed exclusively by an area in which the coating 22 is reduced in thickness. For example, a substrate 1a provided within the measuring body 1 can be used as a flat plane-parallel body without being processed.

FIG. 4 shows a plan view of a substrate 1a, which can be part of a measuring body 1. The substrate 1a is formed as a flat plane-parallel body, for example from silicon, in particular as a wafer, which can be thinner than 1 mm. However, a sandwich structure may also be provided as a substrate, which comprises several wafer layers or a thicker wafer with one or more recesses, in particular etched areas. An optical waveguide structure 6 in the form of an interferometer is applied on or in the substrate 1a. This can be carried out, for example, by the silicon wafer first being covered with a silicon oxide layer and silicon optical waveguides being applied to this. These can in turn be covered with a silicon oxide layer.

The substrate 1a can then be covered as a whole on one or both sides with a protective or functional layer, which can likewise consist of silicon or also a polymer or glass, for example.

The interferometer 12 shown is implemented as a Mach-Zehnder interferometer and has a measuring arm 12a and a reference arm 12b. The detection light generated by the detection light source 5 is routed through an input optical waveguide 6a of the first optical waveguide structure 6 to a beam splitter 6c, where the light is divided into two partial light beams passing through the measuring and reference arm 12a, 12b respectively. The reference arm 12b can have a minimum distance of at least 1 mm or at least 2 mm or at least 5 mm or at least 8 mm from the measuring arm 12a, in order to exclude or reduce as far as possible any influence on the reference arm 12b by an action of the incoming temperature and/or pressure wave. The measuring body 1 is then positioned relative to the excitation light beam 10 in such a way that a temperature and/or pressure wave emitted from the substance to be analysed predominantly reaches the measuring arm 12a of the interferometer and there modifies the refractive index of the optical waveguide.

For example, the two arms of the interferometer can lie in a plane which is parallel to the measuring surface, but also in a plane oriented perpendicular to the measuring surface.

The result is a phase shift between the light beams travelling in the different arms of the interferometer, which leads to a cancellation or partial cancellation of the detection light when the light beams are coupled in the second coupler 6d, depending on the phase position. The intensity of the detection light is then detected by the measuring device 7 in the output optical waveguide 6b of the first optical waveguide structure 6 or at its end or at a coupling point. For example, the detection light can comprise wavelengths in the visible range or also in the infrared range.

Alternatively, instead of an interferometer, an optical waveguide resonance element such as a ring resonator or a plate resonator with an element for coupling in detection light and a decoupling element can be used as a sensor for pressure and/or temperature changes.

FIG. 5 shows an interferometer as a variant of an interferometric assembly, which is combined with an optical waveguide resonance ring 13. This is implemented by the measuring arm of the interferometer being coupled to the resonance ring 13 at two coupling points 13a, 13b. By integrating a resonance ring 13 into one arm of an interferometer, a significantly higher temperature sensitivity of the arrangement can be achieved.

FIG. 6 shows a cross-section through a measuring body 1 with a substrate 1a. A first optical waveguide 15 of a first optical waveguide structure is arranged on the substrate 1a. The first optical waveguide 15 can be integrated on the substrate 1a. Behind the optical waveguide 15 as seen from the measuring surface 2, a heat sink 20 is provided in the form of a body that runs parallel to the optical waveguide 15 above it and is introduced, for example, encapsulated in, the material of the measuring body. The heat sink 20 can also rest directly on top of the optical waveguide 15. The material of the heat sink 20 has a higher specific thermal conductivity and/or a higher specific thermal capacity than the material of the optical waveguide 15 and/or than the material of the substrate 1a and/or than a material with which the substrate 1a is covered.

For example, the first optical waveguide 15 forms a measuring arm of an interferometer. The corresponding reference arm is implemented as a second optical waveguide 16 and integrated on a further substrate 1b, which can either be produced contiguously with the substrate 1a or coupled with it and encapsulated in a common measuring body 1. Between the measuring surface 2, in particular between the substrate 1a, and the second optical waveguide 16 a thermal barrier 30 is arranged, which at least in sections extends parallel to the second optical waveguide 16 between this and the measuring surface and shields it from the action of a pressure and/or temperature wave passing through the measuring surface 2. Alternatively or in addition to the thermal barrier 30, the optical waveguide 16 can be shielded from the area of the measuring surface 2 by a gas gap. Such a gas gap can be introduced into the substrate 1a by etching or another abrasive process, for example, or it can be provided in a casting compound with which the substrate 1a is potted with the measuring body 1. The thermal barrier 30 may also be implemented in the form of a body as a barrier against a pressure wave, and for this purpose have a plasticity or elasticity higher than that of the material of the measuring body 1 that directly surrounds the optical waveguide 16. In many cases and due to the small size of the interferometric elements, it will be useful to implement the thermal barrier by means of trenches etched in a substrate, for example in the substrate 1a or the substrate 1b. For example, the thermal barrier has a conductivity for pressure or thermal waves that is significantly lower than that of a potting material of the measuring body or the substrate 1a, 1b.

FIGS. 6a to 6g show various embodiments of an interferometric device, in each of which the measuring arm and the reference arm are designed in such a way that the effect of a temperature and/or pressure wave on the reference arm with regard to a change in the refractive index is less than the effect on the measuring arm. This is achieved in some cases by positioning the reference arm at a greater distance from the measuring surface 2 than the measuring arm. In some cases, an obstacle or barrier is provided between the reference arm and the measuring surface 2. In other cases, the reference arm is decoupled or spaced apart from the substrate, while the measuring arm is connected to the substrate in a heat-conducting and/or rigid mechanical coupling.

FIG. 6a shows a measuring arm in the form of an optical waveguide 15a and a reference arm in the form of an optical waveguide 16a. A beam divider or splitter is labelled as 35, while a coupler in which the beams of the measuring arm and the reference arm are re-combined is labelled as 36. The reference arm is routed in a central region of the measuring body 1 at a distance D from the measuring arm over a length L. The reference arm is arranged on the side of the measuring arm facing away from the measuring surface 2 and is therefore further away from the measuring surface 2 than the measuring arm by the amount D.

FIG. 6b shows a measuring arm in the form of an optical waveguide 15b and a reference arm in the form of an optical waveguide 16b. Here again, as in the following figures, the beam splitter, which distributes the detection light onto the measuring arm 15b and the reference arm 16b, is labelled as 35 and the coupler as 36. The splitter and coupler can be formed either as a separate optical element or as an element integrated into the substrate of the measuring body 1.

The reference arm 16b is routed in a central region of the measuring body 1 at a distance from the measuring arm 15b of at least the amount D.

Between the measuring arm and the reference arm, a barrier, which is also not shown here, can be provided, which keeps the thermal and/or pressure waves away from the reference arm.

The measuring arm may also have a length greater than the length of the reference arm because the measuring arm, at least in sections, runs in loops and/or has a spiral or meandering shape. However, it may also be provided that the reference arm at least in sections runs in loops and/or has a spiral or meandering shape. Loops, spirals or meandering sections of the measuring arm and/or the reference arm can certainly run in a plane parallel to the measuring surface 2, but also in a plane perpendicular to the measuring surface 2.

FIG. 6c shows a measuring arm in the form of an optical waveguide 15c and a reference arm in the form of an optical waveguide 16c. The measuring arm extends as an optical waveguide which is cast or glued into an opening of the substrate of the measuring body 1 by means of a solid material 37. The material 37 is suitable for conducting thermal and/or pressure waves with as short a delay as possible. For example, the material 37 can be a resin or a polymer. The optical waveguide 15c can be a fibre-optic cable, for example. The optical waveguide 16c forming the reference arm can run along the measuring body 1 without a rigid coupling thereto and be implemented as a fibre-optic cable.

FIG. 6d shows a measuring arm in the form of an optical waveguide 15d and a reference arm in the form of an optical waveguide 16d. The optical waveguide 15d can be integrated into the substrate of the measuring body 1 as an integrated optical waveguide. The optical waveguide 16d can extend on or in the measuring body 1 within an embedded section into a material 38, the material 38 being structured in such a way that it conducts thermal and or pressure waves less well than does the material of the measuring body 1 or the substrate of the measuring body 1. For example, the material 38 can be formed as a silicone, in general as an elastomer and/or foam.

FIG. 6e shows a measuring arm in the form of an optical waveguide 15e and a reference arm in the form of an optical waveguide 16e. Both optical waveguides 15e, 16e extend within the measuring body 1, in particular as optical waveguides integrated into the substrate, but are separated by a barrier layer 39. This consists of a material that conducts thermal and or pressure waves less well than the material of the measuring body 1 or the substrate of the measuring body. For example, the barrier layer 39 can be formed as a silicone, in general as an elastomer and/or foam or from a soft, for example thermoplastic, plastic. The barrier layer 39 can also be implemented as a gas gap, at least in some sections.

FIG. 6f shows a measuring arm in the form of an optical waveguide 15f and a reference arm in the form of an optical waveguide 16f. The measuring arm is arranged between, for example, a slit-shaped opening 40 of the measuring body or a substrate of the measuring body 1 and the measuring surface 2. The reference arm is arranged on the side of the opening 40 facing away from the measuring surface 2. The opening can be implemented as a blind hole, for example, as a bored hole or as a plurality of bored holes. The measuring arm can also have a length greater than the length of the reference arm because the measuring arm, at least in some sections, runs in loops and/or in a spiral or meandering shape above the opening 40. However, as shown in the figure, it may also be provided that the reference arm at least in some sections runs in loops and/or has a spiral or meandering shape.

FIG. 6g shows a measuring arm in the form of an optical waveguide 15g and a reference arm in the form of an optical waveguide 16g. The reference arm is arranged on the side of the slit-shaped opening 41 facing away from the measuring surface 2 and passing through the measuring body 1 perpendicular to the drawing plane. The opening 41 can also be implemented as one or more bored holes, but can also be introduced in a technique commonly used in forming substrates, such as etching technology or laser cutting or other abrading process. Such a substrate can also be formed in an additive process (3D-printing). The measuring arm may also have a length greater than the length of the reference arm because the measuring arm, at least in sections, runs in loops and/or has a spiral or meandering shape. However, as shown in the figure, it may also be provided that the reference arm at least in sections runs in loops and/or has a spiral or meandering shape. The loops, spirals or meanders can each run in a plane parallel to the measuring surface 2, but also in a plane perpendicular to the measuring surface 2.

FIG. 6h shows two optical waveguides 15h, 16h, between which light waves can be coupled by means of the resonance element 17h in the form of an optical waveguide resonance ring. The intensity of a light wave fed into the optical waveguide 15h and transported/overcoupled from the optical waveguide 15h to the optical waveguide 16h or via a further optical waveguide resonance ring 19h to the optical waveguide 18h, measured, for example, by the ratio of the intensities of the light wave decoupled at the optical waveguide 16h or 18h to that coupled into the optical waveguide 15h, depends on how distant the wavelength of the light wave is from a resonance wavelength of the resonance element or of the multiple resonance elements. A pressure and/or temperature wave can detune the resonance element/elements by variation of the refractive index, so that the resonance element/elements represent(s) an efficient temperature and/or pressure sensor. As shown in the figure, a plurality of such elements, for example at least two, at least three or at least five, can also be connected in series to increase the sensitivity.

A parallel connection of a plurality, for example at least two, more than two, more than three or more than five, of such elements 17i, 19i is also conceivable, as shown in FIG. 6i between the input optical waveguide 15i and the output optical waveguide 16i. This also allows the sensitivity of the temperature and/or pressure measurement to be controlled.

When using optical waveguide resonance elements, the temporal profile of the intensity of the detection light can be measured by means of an evaluation device and from this, the temporal profile or waveform of the temperature or the pressure during the passage of pressure and/or thermal waves can be measured. From the temporal profile, which can be periodic when using modulation, the absorption strength of the excitation beam in the substance to be analysed can be determined and a spectrum can be determined from this. For example, the temporal profile or waveform of the intensity of the detection light can be used to evaluate the amplitude or a mean value of the deviation of the intensity with the activated, modulated excitation beam from the intensity with the excitation beam deactivated.

FIG. 6k shows, similarly to FIG. 6a, a measuring arm in the form of an optical waveguide 15a and a reference arm in the form of an optical waveguide 16a. A beam divider or splitter is labelled as 35, while a coupler, in which the beams of the measuring arm and the reference arm are re-combined, is labelled as 36. The reference arm is routed in a central region of the measuring body 1 at a distance of size D from the measuring arm over a length L. The reference arm 16a is arranged on the side of the measuring arm 15a facing away from the measuring surface 2 and is therefore further away from the measuring surface 2 than the measuring arm by the amount D. Reference sign 23 designates an evaluation device that detects and evaluates the light intensity behind the coupler 36 and assigns it a phase shift of the detection light and hence an absorption intensity of the excitation beam in the substance to be analysed.

Inside the measuring body, one or more heat sinks and/or one or more thermal barriers or neither of these can be arranged, so the measuring body 1 can also be homogeneous and free of heat sinks or thermal barriers.

A thermal and/or pressure wave, which propagates from the substance through the measuring surface 2 into the measuring body 1, first strikes the first measuring section (measuring arm 15a) of the interferometer and generates a phase shift of the detection light there. A time t later, which is determined from the propagation velocity of the wave in the measuring body and the distance D, a phase shift is generated in the second measuring arm/reference arm 16a of the interferometer. If both phase shifts persist at the same time over a period of time, the phase shifts cancel out and do not produce any changes in the intensity of the detection light. During the time intervals in which the wave only acts in one arm/measuring section 15a, 16a, the detection light in the first arm followed by the detection light in the other arm either leads or lags. The temporal profile of this sequence of events is predictable due to the known propagation velocity of the wave in the measuring body. The magnitude of the change in the intensity of the detection light detected by the evaluation device 23 allows the determination of the amplitude of the thermal and/or pressure wave and hence the absorption strength of the excitation light in the substance to be analysed.

FIG. 6l shows the intensity characteristic I of the detection light after passing through the interferometric device on the vertical axis, plotted against time on the horizontal axis.

At time t1, the wave arrives in the measuring body on the measuring arm 15a, causing a phase shift of the detection light there relative to the light that arrives via the reference arm 16a. As a result, the intensity drops from I1 to I2. At time t2 the wave reaches the reference arm 16a where it also causes a phase shift of the same magnitude and direction. Since the influence of the wave on the measuring arm still persists, the phase shifts are cancelled out, so that no (partial) cancellation of the light components from the different arms of the interferometric device takes place. The intensity of the detection light reaches the value I1 again after t2.

Then the intensity decreases after t3, since a phase shift is now only present in the reference arm 16a and after t4, that is, after the wave has completely passed through the interferometric device, the intensity I1 occurs again. The difference between I1 and I2 can be used to determine the amplitude of the wave and thus the absorption strength of the excitation beam in the substance.

FIG. 6m shows an arrangement in which the excitation beam 10 from the excitation beam source 4 penetrates into the substance 3 past a boundary surface of the measuring body 1 to be absorbed there, which is indicated by a stylised circle. From there, the thermal and/or pressure wave is released and propagates inter alia into the measuring body 1 and to the interferometric device 12.

In addition, another position of the excitation beam source 4′ is indicated, from which the excitation beam 10′ is irradiated diagonally past the measuring body 1 into the substance 3 and is absorbed underneath the measuring body 1. In this case, an even greater proportion of the wave reaches the measuring body 1 and the interferometric device. Guidance of the excitation beam by means of an optical waveguide is also conceivable. A body (shown by dashed lines) made of another material can be attached to the measuring body 1, which body is, for example, transparent to the excitation beam 10 and in particular more transparent than the material of the measuring body 1.

FIG. 6n shows that the measuring body 1 can be coupled with the substance to be analysed in the area of the measuring surface not only by direct physical contact, but also by interposing a medium such as an intermediate layer of a solid material or a fluid layer or else a gas layer.

FIG. 6n shows the specific case of a recess 200 on the measuring surface 2, which can also be optionally surrounded by a raised edge 201 on the measuring surface 2. Another way to create a cavity is to simply provide a peripheral raised edge on the measuring surface. If the measuring surface 2 is placed in contact with the substance to be analysed, for example a body part of a living organism, a cavity is formed between the substance and the measuring body, which forms an acoustic coupling element. The pressure and/or thermal wave can enter the cavity from the substance and enter the measuring body through a gaseous medium, where the wave can be detected by an interferometric element 6. Due to the high sensitivity of the interferometric measuring method, the wave can thus also be effectively detected acoustically and its intensity measured.

The excitation beam can be routed directly from the excitation beam source 4 through the cavity 200 to the substance to be analysed. For this purpose, the measuring body can at least partially comprise an opening for the excitation beam, or the latter can be guided through the measuring body by means of an optical waveguide. The excitation beam can also be at least partially guided through the material of the measuring body 1.

FIG. 7 shows a variant of the measuring body 1 in which the optical waveguides 15, 16 of the interferometric arrangement are arranged on the side of a substrate 1a facing the measuring surface 2. On this side, the substrate 1a is covered with a coating 42 that covers and protects the optical waveguides 15, 16. By means of this arrangement, at least one of the optical waveguides 15, which represents the measuring arm of the interferometric arrangement, can be reached directly by a temperature and/or pressure wave from the substance 3. The reference arm 16 should be shielded from the effect of the pressure and/or temperature wave by means that are not shown. For example, the reference arm 16 can be located sufficiently far away from the measuring arm 15 to be significantly less influenced by the effect of a pressure and/or temperature wave than the measuring arm.

FIG. 8 shows a variant in which an interferometric arrangement is realized with fibre-optic cables 15′, 16′, which are firmly connected to the substrate. In the example shown, the connection is implemented by an adhesive 14. The optical waveguides can run in grooves of the measuring body/substrate.

FIG. 9 shows a cross-section through a measuring body 1, which has a continuous opening 18 in the form of a bored hole through which the excitation light beam 10 can pass in a straight line and enter the substance 3 to be analysed. If the measuring body 1 is provided with a coating 22 on its underside, as shown in FIG. 1, the opening 18 can end at the coating, provided that the coating is transparent to the excitation beam 10. The opening 18 can also completely penetrate the coating 22, however.

For example, a beam-shaping element in the form of a lens or a collimator 31 can be provided in the opening 18. The beam guidance of the excitation beam shown in FIG. 9 can be combined with any type of interferometric device (not shown in FIG. 9) shown in the figures and described above.

FIG. 10 shows an arrangement in which the laser device 4 irradiates the excitation light beam 10 directly into the measuring body 1 parallel to the measuring surface 2. A continuous opening 18′ in the measuring body 1 is provided, which is bent at right angles towards the measuring surface 2. In the area of the change of direction, a reflection element 32 is provided, for example in the form of a mirror. In the arrangement shown, the excitation light beam 10 can enter the substance to be analysed 3 through the measuring surface 2 at right angles. The beam guidance of the excitation beam shown in FIG. 10 can be combined with any type of the interferometric devices (not shown in FIG. 10) shown in the figures and described above.

FIG. 11 shows a cross-section through a measuring body 1, in which a second optical waveguide structure 17 is provided for guiding the excitation light beam 10. This can be designed as an integrated optical waveguide which is integrated into a substrate of the measuring body 1. The second optical waveguide structure 17 is aligned such that the excitation light beam 10 is guided perpendicularly through the measuring surface 2. However, it is also conceivable that the optical waveguide of the second optical waveguide structure 17 is directed at the measuring surface 1 at an angle of less than 90°, for example less than 700 or less than 50°. The laser device 4 can be coupled to the second optical waveguide structure 17 directly or by interposing a beam-shaping element, for example a lens (not shown in FIG. 11), but a flexible fibre-optic cable may also be provided for guiding the excitation beam 10 between the laser device 4 and the second optical waveguide structure 17. The beam guidance of the excitation beam shown in FIG. 11 can be combined with any type of interferometric device (not shown in FIG. 11) shown in the figures and described above.

At the end of the integrated optical waveguide of the second optical waveguide structure 17 facing the measuring surface 2, a beam-shaping element, for example a lens (not shown in FIG. 11), can also be provided.

FIG. 12 shows a more complex shaped integrated optical waveguide 17a within the second optical waveguide structure 17, which guides the excitation light beam 10. The excitation light beam 10 is coupled, for example, parallel to the measuring surface 2 into the integrated optical waveguide 17a of the second optical waveguide structure 17 and redirected by this integrated optical waveguide 17a in a direction that passes through the measuring surface 2, in particular one passing through at right angles or else at an angle of less than 90°, for example, less than 70° or less than 50°. In the substrate 1a, a modulation device 8 is integrated in the region of the second optical waveguide structure 17, which performs the intensity modulation of the excitation light beam by means of the processing device 23. The modulation device 8 can be implemented, for example, by a piezoelement arranged in or on the second optical waveguide structure 17, or by a heating element that modulates the transparency of the second optical waveguide structure 17, or by a MEMS mirror element for deflecting the excitation light beam 10.

The integrated optical waveguide 17a of the second optical waveguide structure 17, which guides the excitation beam 10, has sections in which it runs parallel to the measuring surface and sections in which it runs in a direction towards the measuring surface 2, in particular at right angles to the measuring surface 2. Forming such an optical waveguide in a substrate 1a is possible in a proven manner using means from the field of integrated optics.

FIG. 13 schematically shows the processing of measurement data obtained with the device for analysing a substance. In the left-hand part of FIG. 13, a measuring body 1 and a laser device 4 for generating an excitation beam are shown, as well as a measuring device 7. The measuring device 7 and in particular also the laser device 4 are connected to the processing device 23, which can be implemented as a microcontroller or as a microcomputer and comprises at least one processor. In the processing device, the measurement data of the variable light intensity acquired by the measuring device 7 are combined or correlated with the data of the modulated excitation beam, i.e. with the respectively emitted wavelengths and the temporal waveform of the modulation. Three diagrams are shown in symbolic form, the top one of which shows the modulated laser pulses plotted against time, while the middle diagram shows the temporal waveform of the measurement data. Each time a thermal and/or pressure wave arrives at the interferometric element, for example, by activating/deactivating or modulating the excitation beam, the element is detuned by a change in the refractive index, or the wave components from different measuring arms of an interferometer are cancelled or partially cancelled. This changes the intensity of the detection light after it has passed through the interferometric element. This temporal profile or waveform, in addition to showing the absorption strength of the excitation beam in the substance to be analysed, also reflects the mixing characteristics of the signals which are sent to the device for each period of modulation of the excitation beam as a mixture of signals from different depths below the substance surface and which, due to the transit time differences, produce a specific decay characteristic of the measuring signals after each laser pulse. The signals from different depths do not need to be separated from each other, but this can be carried out using different analysis methods which are explained elsewhere in this text.

The third and bottom diagram shows a spectral plot in which measured light intensities are plotted over the irradiated wavelengths or wave numbers of the excitation light beam in a series of spectra.

For example, these data can be used to obtain physiological values of a patient, which are obtained from measurement of the concentration of certain substances in the body tissue or in a bodily fluid. An example of this is blood glucose measurement, which measures the glucose concentration in a part of the body. According to FIG. 13, measured values or pre-processed values can be compared using a remote computer or a distributed computer system (cloud) by means of a communication device 25. For example, reference values can be imported from the cloud or a remote computer to interpret the measured values. The reference values can be based on the identity of the patient and data that can be stored and retrieved individually for him/her. For this purpose, the identity of the patient must either be entered in the processing device 23 or it must be determined by means of separate measurements, for example by means of a fingerprint pressure sensor which can be integrated into the measuring device.

Within the cloud, it is possible also to compare the data with measurements from other patients or with previous measurements from the same patient, including taking account of environmental conditions such as temperature, air pressure, or air humidity at the patient's location. Sensors for acquiring these values can be integrated into the apparatus/device for analysing a substance according to the invention.

As a processing result, the processing device 23 can output trend information, for example in three or five levels, in the form of information such as optimum, good, reasonable, could be better, concerning, or in the form of colours or symbols, using a first output device 26. In another output device 27, which allows a specific value display, measured values can be output on a screen or in a digital display. In addition, measured values or measured value trends can be output to a software module 28, which can also run, for example, in a separate mobile processing device such as a mobile phone. In this unit, the evaluated measurement results can then be used, for example, to prepare a meal to be taken or to select available foodstuffs and a quantity of food. Also, a recommendation can be made for the consumption of certain foods in a particular quantity. This can be linked, for example, to a proposal for preparation, which can be retrieved from a database and, in particular, also transmitted in electronic form. This preparation instruction can also be sent to an automatic food preparation device.

In one embodiment, a suggestion for an insulin dose depending on other patient parameters (e.g. insulin correction factor), or an automatic signal transmission to a dosing device in the form of an insulin pump, can be output via the display device/display 27 or a signal device parallel to this.

The processing device 23 can be integrated into the housing 33 of the device, but it can also be provided separately, for example in a mobile computer or a mobile wireless device. For this case, provision must be made for a communication interface between the components arranged in the housing 33, in particular the measuring device 7 and the processing device 23, for example using a radio standard. The housing 33 can be designed as a wearable case, for example also as a case that can be worn on a person's wrist in the manner of a wristwatch (wearable). In a further embodiment, the laser device can also be arranged outside the housing and designed to be coupled for a measurement. The coupling can be implemented, for example, by means of a fibre-optic cable and/or by suitably aligning the excitation beam of the laser device by applying the laser device to a reference surface of the housing relative to the measuring body for a measurement.

FIG. 14 shows a plan view of a substrate 100, which carries an excitation light source 4, for example in the form of a laser device, in particular a laser array. In addition, the substrate 100 carries a detection light source 5 and a measuring device 7, for example in the form of a radiation-sensitive semiconductor component for measuring the intensity of the detection light. Each of the elements 4, 5, or 7 can also be integrated completely or partially into a semiconductor structure of the substrate 100 or be produced from it by micromechanical manufacturing technology and doping, for example. The substrate comprises optical waveguides 101, 102, 103, which are either completely or partially integrated into the substrate or are fixed to it in the form of fibre-optic cables, for example in V-grooves, which position the optical waveguides sufficiently accurately. The optical waveguide 101 guides the excitation light/excitation radiation, while the optical waveguides 102, 103 guide the detection light/detection radiation.

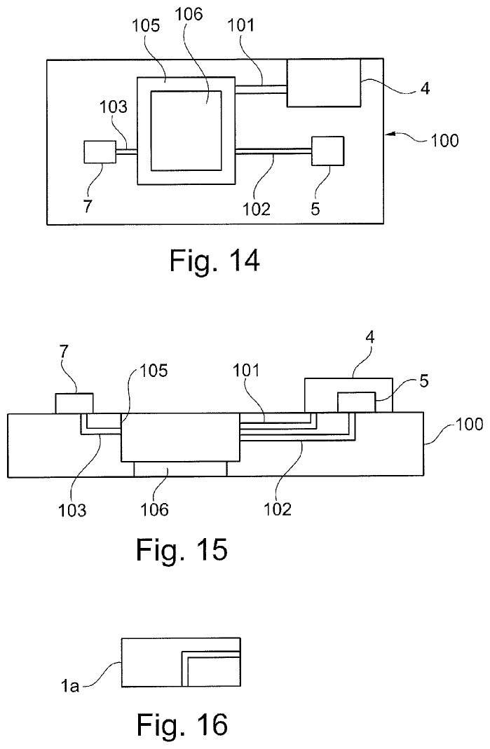

The substrate 100, as can be seen particularly clearly in FIG. 15, has a precisely micromechanically fitted opening 105 into which another substrate 1a can be fitted in such a way that one or more of the optical waveguides 101, 102, 103 end directly in front of corresponding connecting optical waveguides (not shown) of the other substrate, so that the guided radiation can be directly coupled into the optical waveguides of the other substrate 1a and then decoupled from them toward the optical waveguide of the measuring device/the detector 7. Coupling elements can also be provided for this purpose, which increase the efficiency of the coupling. As shown in FIG. 16 and in the comparable FIG. 12, the substrate 1a then comprises an integrated optical waveguide which guides the excitation light toward the measuring surface. In addition, the substrate 1a has an integrated interferometric element with integrated connecting optical waveguides. The measuring surface can be located on either side of the substrates 100, 1a. If the measuring surface is located on the lower side in FIG. 15, a window 106 can be provided within the opening 105 as a continuous opening in the substrate 100.

FIG. 17, like FIGS. 18 to 22, shows a cross-sectional view of a substrate 1a into which a first optical waveguide arrangement 6 is embedded as part of a detection device. Hatched areas are omitted in cut portions for the sake of clarity. The measuring surface 2 is located in the upper part of the substrate 1a in each figure. For illustration purposes, in FIG. 17 as in FIG. 20 a human finger 107 is shown as an example of a measurement object, the substance of which is to be analysed. The finger is placed on the measuring surface 2 for analysis.

FIGS. 17 to 22 each show substrates, the material of which is permeable or at least partially permeable for an excitation beam 10 in the infrared region or in general in the wavelength range of the excitation beam. For example, this applies to a silicon substrate for the infrared range. An excitation beam can therefore be directed through the substrate material, or at least through limited layer thicknesses, onto the measuring surface and through this into the substance to be measured. In such a case, it is not necessary to provide a continuous opening in the substrate for the excitation beam 10. The excitation beam can be directed past or through the first optical waveguide structure 6. Part of the distance travelled by the excitation beam in the measuring body can also be inside an opening/cavity. For this purpose, an opening can be provided at least in some sections of the measuring body. For example, a thin layer of the substrate can then remain in place in the area of the measuring surface. However, in sections of the measuring body a material insert may be provided in the form of an optical waveguide made of a material that is more transparent to the excitation beam than the material of the substrate.

In FIGS. 17-22 and 23-25 various configurations for the guiding of the excitation beam unit are shown. The detection device is omitted in each case for clarity. Of course, all the described designs of the detection device can be implemented in combination with the designs of the excitation beam guidance shown in FIGS. 17-25.

On the side of the measuring body or the substrate 1a opposite the measuring surface 2, a lens 108, 108′, 108″ is integrated into the substrate, formed in particular by the material of the substrate and extracted from the material of the substrate, for example using abrasive methods, in particular by etching.

Three examples of possible lens shapes are shown in FIGS. 17 to 22, the first lens being shown in FIGS. 17 and 20, the second lens in FIGS. 18 and 21 and the third lens in FIGS. 19 and 22.

The first lens 108 corresponds to a normally refracting, refractive convex convergent lens, the second lens 108′ corresponds to a (refractive) convergent lens ground to the Fresnel form (Kinoform lens), and the third lens corresponds to a diffraction lens, which focusses the excitation beam 10 by diffraction at a concentric lattice structure.

The optical axes of the lenses can each be positioned perpendicularly on the measuring surface 2, so that an excitation light source can radiate directly straight through the substrate 1a. However, the optical axes can also be inclined with respect to the perpendicular to the measuring surface 2 in order to allow a potentially space-saving positioning of the excitation light source at an angle to the substrate.

FIGS. 20, 21 and 22 each show the lens shapes 108, 108′, 108″ on the substrate 1a with the excitation beams 10 and the focused beams 10a focused on the substance to be analysed.

In FIGS. 17-22, interferometric elements are provided in the substrate near the measuring surface in each case, however, in these figures the main intention is to show the beam guidance of the excitation beam 10.

FIG. 23 shows a measuring body 1 with a sensor layer 1′ in cross-section, in which an excitation beam 10 is directed out of the laser arrangement 3 into an optical waveguide 126, which passes through the measuring body 1 to the layer 1′. The optical waveguide 126 can also extend through the layer 1′ as far as the measuring surface 2, but it may also be provided that either the layer 1′ has a slot for the excitation beam 8 or the excitation beam 8 passes through the material of the layer 1′. Also, a certain layer thickness of the substrate, which in the exemplary embodiment shown forms the measuring body 1, can remain in place in front of the measuring surface or in front of the layer 1′ and be traversed by the excitation beam 10. In the region of measuring surface 2, for example directly adjoining the measuring surface 2 and/or within the layer 1′, a lens 140 can be provided to focus the excitation beam 10 on a point in the substance to be examined. The optical waveguide 126 runs in a straight line from the laser device 4 to the measuring surface 2 and passes through the detection device formed by an interferometric element, not shown in detail, in the layer 1′ or in the substrate 1. An optical waveguide can also run partially or completely along the surface of the measuring body 1, for example, if the laser device 4′ is positioned at the side of the measuring body (see FIG. 25). In FIG. 23, the optical waveguide 127 runs firstly from the laser device 4′ on a first part of its length at or on the surface of the measuring body, in order then, like the optical waveguide 126, to continue to pass through the measuring body over a second part of its length. In the region of the change of direction of the optical waveguide the excitation beam can be reflected, for example at a mirror, or the optical waveguide can be bent there. Such an optical waveguide 126, 127 can be integrated into the material of the measuring body 1 by manufacturing techniques (e.g. using SOI—Silicon on insulator technology), or in the form of a fibre-optic cable connected thereto by adhesive bonding, for example, or the optical waveguide can be integrated over part of its length and implemented as a fibre-optic cable over another part of its length.

However, as can be seen from FIG. 24 in two different variants of the optical waveguide design, a curved optical waveguide 133, 134 can also be provided, which guides the excitation beam 10 from a position on the measuring body 1 at which the laser device is provided toward the measuring surface 2. The fact that the route of the optical waveguide 133, 134 can be shaped relatively freely allows a minimum distance to be maintained between the region penetrated by the excitation beam and the detection region. The excitation beam 10 can also strike the measuring surface 2 at an angle between 0 degrees and 60 degrees, in particular between 0 and 45 degrees to the surface normal of the measuring surface 2, and pass through it.

Due to the low penetration depth into the substance to be analysed, despite an oblique irradiation direction the region of the substance in which the excitation beam 10 interacts with it lies directly below the detection device, which can be in the form of an interferometric element, for example. For example, at least some sections of the curved optical waveguides 133, 134 can be laid as fibre-optic cables in a bored hole or similar recess of the measuring body 1, where they are glued or potted in place.

As can be seen from FIG. 25, an optical waveguide 135, 136, 137, 138 can also be provided for guiding the excitation beam 10, which is routed, for example, in multiple directions and/or in two or three mutually perpendicular directions along one, two or three different, mutually adjacent surfaces of the measuring body 1. For example, such an optical waveguide 135, 136, 137, 138 can be integrated into the respective measuring body 1, as can the optical waveguides shown in FIGS. 23 and 24. On the surfaces of a measuring body, this is particularly simple to implement in SOI technology or, depending on the material of the measuring body 1, in a related solid-state manufacturing technology. For this purpose, an optical waveguide can be incorporated in a silicon substrate, which is covered and separated from the substrate by silicon oxide layers or other layers. To this end, a suitable recess can first be etched or sputtered into the substrate, in order then to suitably deposit the material of the covering and the optical waveguide. In this case, for example, the covering of the optical waveguide can be aligned flush with the surface of the measuring body so that the optical waveguide does not protrude beyond the measuring body 1. The course of the optical waveguide 135, 136, 137, 138 along the surfaces of the measuring body prevents any interaction of the excitation beam with the detection device. The last optical waveguide 138 then ends in the region in which the excitation beam 10 should enter the substance 3 to be analysed. At the end of the optical waveguide 138, an element can be provided there, for example a mirror, that directs the excitation beam into the substance 3.

The detail shown in FIG. 25 in an area 142 in the lower right section of the figure shows that the optical waveguide 138 can also be arranged in a groove (shown dotted) of the measuring body 1 leading diagonally onto the measuring surface 2, so that the longitudinal axis of the optical waveguide is oriented parallel to the bottom 141 of the groove through the measuring surface 2 and onto the substance 3 to be analysed.

The present patent application relates (as already mentioned at the outset) to the following aspects in addition to the subject matter of the claims and exemplary embodiments described above. These aspects, or individual features thereof, can be combined with features of the claims, either individually or in groups. The aspects also constitute independent inventions, whether taken in isolation or combined with one another or with the subject matter of the claims. The applicant reserves the right to make these inventions the subject of claims at a later date. This may take place within the scope of this application or in the context of subsequent part applications or subsequent applications, claiming the priority of this application.

### Aspects

1) Method for analysing a substance in a body, comprising:
emitting an excitation light beam (excitation beam) with one or more specific excitation wavelengths through a first region of the surface of the body,
intensity modulation of the excitation light beam with one or more frequencies, in particular sequentially, by means of a mechanical, electrical or optical chopper, in particular by an electronic activation of the excitation light source, an adjustment device for a resonator of an excitation laser acting as an excitation light source or a movable mirror device, a controllable diffraction device, a shutter or mirror device coupled to a motor, such as a stepper motor, or to a MEMS, or a layer in the beam path that can be controlled with respect to transmission or reflection, and time-resolved detection of a response signal
by means of a detection device arranged outside the body, the response signal being due to the effect of the wavelength-dependent absorption of the excitation light beam in the body and the emission of a temperature and/or thermal wave to the detection device.

The detection device may comprise, for example, an optical medium/measuring body with a detection region, which is in particular adjacent to or directly adjoining the measuring surface (which corresponds to the boundary surface of the measuring body in contact with the substance to be analysed), and which, in the event of pressure or temperature changes, affects a detection light beam that passes the measuring body by changing its refractive index. In particular, the intensity of the detection light can be influenced by the changes in pressure and/or temperature.

For example, the detector/detection device may have an optical waveguide integrated on a substrate, in particular in “Silicon on insulator” technology. For example, silicon is used for the optical waveguide. The use of SiN is also possible, wherein the optical waveguide should be at least partially covered by a silicon oxide which has a different refractive index from the refractive index of Si or SiN.

The modulation can be carried out in one embodiment by interference or by manipulating the phase or polarization of the radiation of the excitation transmission device, in particular if this comprises a laser light device. The modulation can also be performed by controlling an actively operated piezoelement, which is a part or element of the measuring body and the transmission or reflection property or reflectivity of which can be controlled by a voltage controller on the piezoelement. The response signals can be, for example, intensities or deflection angles of a reflected measurement beam or voltage signals of a detector operating with a piezoelectric effect.

2) Method according to aspect 1, characterized in that the excitation light beam/excitation beam is generated by a plurality of emitters or multi-emitters, in particular in the form of a laser array, which emit light at different wavelengths simultaneously or sequentially or in pulse patterns, and also alternately.
3) Method according to either of aspects 1 or 2, comprising the steps:
producing a contact between an optical medium/measuring body and a substance surface of the body, so that at least one region of the surface of the measuring body (e.g. a measuring surface) is in contact with the first region of the surface of the body;
emitting an excitation light beam with an excitation wavelength into a volume located in the substance below the first region of the surface, in particular through the region of the surface of the optical medium, which is in contact with the first region of the substance surface,
measuring the temperature or temperature change and/or a pressure change in the first region of the surface of the measuring body by means of an optical method,
analysing the substance using the detected temperature increase as a function of the wavelength of the excitation light beam. This process can be performed during one measurement for different modulation frequencies and the results for different modulation frequencies can be combined.
   4) Method according to any of the aspects 1 to 3, characterized in that the detection light beam is generated by the same light source that produces the excitation light beam.
   5) Method according to aspect 1 or any of the others preceding or following, characterized in that the excitation light beam is an intensity-modulated, in particular pulsed excitation light beam, in particular in the infrared spectral range, wherein the modulation rate is in particular between 1 Hz and 10 kHz, preferably between 10 Hz and 3000 Hz.
   6) Method according to aspect 1 or any of the others preceding or following, characterized in that the light of the excitation light beam(s) is/are generated simultaneously or sequentially or partially simultaneously and partially sequentially, by means of an integrated arrangement having a plurality of individual lasers, in particular a laser array.
   7) Method according to aspect 1 or any of the others preceding or following, characterized in that from the response signals obtained at different modulation frequencies of the excitation light beam an intensity distribution of the response signals is determined as a function of the depth below the surface at which the response signals are generated.
   8) Method according to aspect 1 or any of the others preceding or following, characterized in that from the phase position of the response signals, i.e. the temperature and/or pressure characteristic in the substance to be analysed, measured by the intensity characteristic of the detection light in relation to the phase of the modulated excitation light beam at one or different modulation frequencies of the excitation light beam, an intensity distribution of the response signals is determined as a function of the depth below the surface at which the response signals are generated.
   9) Method according to aspect 7 or 8, characterized in that to determine the intensity distribution of the response signals as a function of the depth below the surface, the measurement results at different modulation frequencies are weighted and combined with each other.
   10) Method according to aspect 7, 8, or 9, characterized in that a substance density of a substance that absorbs the excitation light beam in specific wavelength ranges at a specific depth or in a depth range is determined from the intensity distribution over the depth below the surface of the body.
   11) Method according to aspect 1 or any of the others preceding or following, characterized in that immediately before or after or during the detection of the response signal/signals, at least one biometric measurement is carried out on the body in the first region of the surface in which the substance analysis is performed or directly adjacent thereto, in particular a measurement of a fingerprint, and the body, in particular a person, is identified and that, in particular, associated reference values (calibration values) are assigned to the detection of the response signals by the identification of the person.

The biometric measurement can also include the measurement of a spectrum of response signals when scanning over a spectrum of the excitation light beam. By evaluation of the spectrum, a profile of substances present in the body and their quantity or density ratio can be determined, which can enable the identification of a person.

12) Apparatus for analysing a substance,
having a device for transmitting one or more excitation light beams, each of which has an excitation wavelength, into a volume located in the substance below a first region of its surface, with a device for modulating an excitation light beam which is formed by a modulating device of the radiation source, in particular the control thereof, an interference device, a phase or polarization modulating device and/or at least one controlled mirror arranged in the beam path, and/or a layer that can be controlled with regard to its transparency and arranged in the beam path, and having a detection device for detecting a time-dependent response signal as a function of the wavelength of the excitation light and the intensity modulation of the excitation light, and having a device for analysing the substance using the detected response signals.
13) Apparatus according to aspect 16, having a device for determining response signals separately according to different intensity modulation frequencies and/or having a device for determining response signals as a function of the phase offset of the respective response signal relative to the phase of modulation of the excitation light beam, in particular as a function of the modulation frequency of the excitation light beam.
14) Apparatus for analysing a substance as defined in 12 or 13, having an optical medium/measuring body for making the contact between the surface of the optical medium (for example, a so-called measuring surface) and a first region of the substance surface, and having
a device for emitting an excitation light beam with one or more excitation wavelengths into a volume in the substance below the first region of the surface, in particular through the region of the surface of the optical medium (the measuring surface) which is in contact with the substance surface, and having a device for
measuring response signals in the form of temperature and/or pressure changes in the region within the measuring body in the immediate vicinity of the measuring surface (using a detection device), which is in contact with the first region of the substance surface, by means of an optical method that makes use of a detection light beam, and having a device for analysing the substance using the detected response signals in the form of temperature changes/pressure changes as a function of the wavelength of the excitation light beam and the intensity modulation of the excitation light beam, in particular the modulation frequency of the excitation light beam.
15) Apparatus according to aspect 18, characterized in that the excitation light source and/or the detection light source is directly mechanically firmly connected to the measuring body.

The excitation light source and/or the detection light source can each be directly coupled to an optical waveguide of a first or second optical waveguide structure, which is provided in, on or on top of the measuring body and can be integrated into it. The excitation light source and/or the detection light source can also be connected to a first or second optical waveguide structure of the above type by means of a fibre-optic optical waveguide in each case.

16) Apparatus according to aspect 18, 19 or 20, characterized in that the measuring body directly carries a beam-shaping lens and/or that a beam-shaping lens is integrated into the measuring body.
17) Apparatus according to any of the aspects 12 to 16, characterized in that the apparatus comprises a wearable housing which can be attached to a person's body, wherein the device for emitting one or more excitation light beams and the detection device for detecting a time-dependent response signal are arranged and configured in such a manner that in operation, when the device is worn on the body, the substance to be analysed is measured on the side of the housing facing away from the body, in particular, that the measuring surface of the measuring body is located on the side facing away from the body.
18) Apparatus according to any of the aspects 12 to 16, characterized in that the apparatus has a wearable housing that can be attached to the body of a person and that the housing of the device has a window that is transparent to the excitation beam on its side facing away from the body in the intended wearing position.

The window can be located directly in front of the measuring body. The window can be a single opening in the housing, the window surface being formed by the measuring surface or the measuring surface being in the opening. The measuring surface can also lie behind a layer that closes the window opening and is connected to the measuring surface in such a way that temperature and/or pressure waves are transmitted from the outside to the measuring surface.

19) Apparatus for analysing a substance with an excitation transmission device for generating at least one electromagnetic excitation beam, in particular excitation light beam, with at least one excitation wavelength, a detection device for detecting a response signal, and a device for analysing the substance using the detected response signal.
20) Apparatus according to any of the preceding aspects 12 to 19, characterized in that the excitation transmission device contains a probe laser or an LED, for example an NIR (near-infrared) LED.
21) Apparatus according to any of the preceding aspects 12 to 20, characterized in that the excitation transmission device has a probe laser, which has a smaller beam diameter than an additional pump laser which forms the laser for generating the excitation beam.
22) Apparatus according to any of the preceding aspects 12 to 21, characterized in that the apparatus is designed to be permanently wearable for a person on the body, in one embodiment by means of a retaining device connected to the housing, such as a belt, a strap or a chain or a clasp, and/or the detection device has a detection surface which also serves as a display surface for information such as measurements, times of day and/or textual information.

The detection surface can be identical to the measuring surface or form its extension/continuation.

23) Apparatus according to the previous aspect 22, characterized in that the apparatus has a peel-off film in the region of the detection surface/measuring surface, preferably next to the detection surface/measuring surface, for pre-treatment of the surface of the substance and ensuring a clean surface and/or, in one embodiment in the case of glucose measurement, specifically for skin cleansing.
24) Apparatus according to the previous aspects 12 to 23, characterized in that the detection device is configured for reading and recognizing fingerprints to retrieve specific values/calibrations of a person and/or that it has a device for detecting the position of a finger, preferably for detecting and determining an unwanted movement during the measurement.
25) Apparatus according to any of the previous aspects 12 to 24, characterized in that the detection device has a result display, preferably implemented with colour coding, as an analogue display, in one embodiment including error indication (e.g.: “100 mg/dl plus/minus 5 mg/dl”), acoustically and/or with a result display of measurement values in larger steps than the measuring accuracy of the device allows (e.g. using a multi-coloured traffic light display). This means that the user is not informed of e.g. small fluctuations, which could unsettle them.
26) Apparatus according to any of the preceding aspects 12 to 25, characterized in that the apparatus
comprises data interfaces for exchanging measured data and for retrieving calibration or identification data or other data from other devices or cloud systems, for example, wired or wireless interfaces (infrared, light or radio interfaces),
wherein the apparatus is preferably configured to ensure that data transmission can be encrypted, in particular encrypted by fingerprint or other biometric data of the user.
27) Apparatus according to any of the previous aspects 12 to 26, characterized in that the apparatus is configured such that a proposal for an insulin dose to be given to the person or substances/foodstuffs including the quantity to be consumed can be determined by the apparatus (e.g. insulin correction factor) and/or that the body weight, body fat can be measured and/or entered manually or transferred from other devices to the apparatus at the same time.
28) Apparatus according to any of the previous aspects 12 to 27, characterized in that to increase the measuring accuracy the apparatus is configured to determine further parameters, in one embodiment by means of sensors for determining the skin temperature, diffusivity/conductivity/moisture level of the skin, or to measure the polarization of the light (excluding water/sweat on the finger surface).

Water and sweat on the surface of a person's skin, which can affect the glucose measurement, can be detected by a test excitation with an excitation radiation by means of the excitation transmission device with the water-specific bands at 1640 cm⁻¹ (6.1 μm) and 690 cm⁻¹ (15 μm) and taken into account in a subsequent analysis of the measurement. Alternatively, the electrical conductivity of the substance can be measured near to or directly at the measuring site using a plurality of electrodes to determine the moisture level. An error message and an instruction for drying can then be issued or the presence of moisture can be taken into account in a subsequent evaluation of a measurement.

29) Apparatus according to any of the preceding aspects 12 to 28, characterized in that the apparatus has a covering or blocking device in the beam path of the pumped and/or measuring beam laser. This can ensure the obligatory eye safety of human beings.
30) Apparatus according to any of the preceding aspects 12 to 29, characterized in that the apparatus has a replaceable detection surface/measuring surface.
31) Apparatus according to any of the preceding aspects 12 to 30 characterized in that the apparatus has a locally corrugated crystal as a measuring body or a crystal provided with parallel grooves or distributed depressions or elevations or is roughened as a measuring body, which allows a better adjustment of the sample (e.g. the finger). The measuring point on which the surface of the substance to be analysed is placed is preferably designed without grooves and smooth.
32) Apparatus according to any of the preceding aspects 12 to 31, characterized in that the apparatus measures not only at one point, but in a grid pattern. This can be carried out either by displacing the pump or probe laser or the detection unit relative to the skin surface of a subject or by a variable deflection of the excitation beam between two measurements.

In addition, the following aspects of the invention are also to be cited:

33) Apparatus for analysing a substance, in particular also according to any of aspects 12 to 32, having
an excitation transmission device/laser device for generating at least one electromagnetic excitation beam, in particular excitation light beam, with at least one excitation wavelength,
a detection device for detecting a response signal and
a device for analysing the substance using the detected response signal.

The time-dependent response signal can take the form of the temperature or pressure increase in the measuring body as well as any measured variable that detects the same, for example the intensity change of detection light which passes through a material with a temperature- or pressure-dependent refractive index.

34) Method for analysing a substance, wherein in the method
using an excitation transmission device/laser device, at least one electromagnetic excitation beam with one or more excitation wavelengths is generated and transmitted into the substance by the at least partially simultaneous or consecutive operation of a plurality of laser emitters of a laser light source,
a response signal is detected with a detection device, and
the substance is analysed on the basis of the detected response signal.
   35) Method according to aspect 34, characterized in that using different modulation frequencies of the excitation transmission device, response signals, in particular temporal response signal waveforms, are successively determined and that a plurality of response signal waveforms at different modulation frequencies are combined with each other and that from this, information specific to a depth range below the surface of the substance is obtained.
   36) Method according to aspect 35,
   characterized in that
   response signal waveforms at different modulation frequencies are determined for different wavelengths of the excitation beam and, in particular, from this information specific to a depth range below the surface of the substance is obtained.
   37) Method according to aspect 36,
   characterized in that
   when using multiple modulation frequencies of the excitation beam at the same time, the detected response signal is separated according to its frequencies by means of an analysis method, preferably a Fourier transform, and
   only one partial signal at a time is filtered, measured and analysed that corresponds to a frequency to be processed.

In this way, a plurality of signals at different modulation frequencies can be analysed successively and the results of different modulation frequencies can be combined with one another to obtain depth information about the signals, or to eliminate signals coming from the surface of the substance.

38) Method according to any one of the preceding aspects 34 to 37, in which as a function of a concentration of the substance determined in the substance, a dosing device is activated to release another substance into the substance, in particular into a patient's body, and/or an acoustic and/or optical signal is emitted and/or a signal is issued to a processing device via a radio link and/or that one or more foodstuffs or foodstuff combinations are assigned to the measured substance concentration by means of a database and output as nutritional information, in particular as a nutritional recommendation.

In addition to or in combination with such a recommendation, a quantity indication can also be given for the foodstuffs or foodstuff combinations. Foodstuff combinations is also intended to mean prepared food portions.

All features and measures of the excitation beam, its optical guidance and modulation, which are mentioned in the aspects in connection with any given measuring method, in particular in connection with a measurement light beam and the detection of its deflection, as well as the features of the mechanical structure and the adjustability, the features of the housing and the communication with external devices, databases and connected devices, can also be applied to the detection method as claimed in the patent claims of the present application, i.e. using an interferometric effect to detect the pressure and/or thermal wave emitted from the substance into a measuring body as a response signal.

In principle, values of a phase shift of the response signal determined for depth profiling in response to a periodic modulation of the excitation beam can be used. (Heating/cooling phases of the substance surface should be evaluated more precisely with regard to their characteristics).

The apparatus described may be connected to a supply of adhesive strips for the removal of dead skin layers in order to allow the best possible interference-free measurement on a human body, as well as patches or other pharmaceutical forms of a coupling medium, in particular a gel or thermal conductive paste, which can be regularly applied to the optical medium. The optical medium may be interchangeable given appropriate mounting and calibration of the remaining parts.

Data acquisition (DAQ) and lock-in amplifiers in the evaluation can be combined in one device and the entire evaluation process can be digitized. The lock-in amplifier is connected to the detection device and selects the signals that are in a phase relationship to the modulation of the excitation beam. For this purpose, the lock-in amplifier is connected, for example, to the control device for the laser device which generates the excitation beam and/or to the modulation device for the excitation beam.

The measurement can also be carried out with the apparatus on a substance surface that is moved relative to it, so that during a grid measurement an excitation light source and/or a measuring light source travel or travels over the skin in a grid pattern during the measurement, thereby compensating for or eliminating skin irregularities.

An additional configuration and explanation of the invention according to the patent claims is presented in the following concept. Details of this concept can also be combined with embodiments of the patent claims in the form in which they were filed. In addition, this concept, whether taken in isolation, combined with the above aspects or with the subject matter of the claims, constitutes at least one independent invention. The applicant reserves the right to make this invention or inventions the subject of claims at a later date. This may take place within the scope of this application or in the context of subsequent sub-applications or follow-up applications that claim priority of this application.

The following concept for non-invasive blood sugar measurement by determining the glucose in the skin by stimulation by quantum cascade lasers and measuring the thermal wave due to radiant heat shall also be included in the invention and can be combined with the objects of the claims or pursued independently in a sub-application:

A method is described that allows the concentration of glucose or any other substance in the interstitial fluid (ISF) in the skin to be determined. Glucose in the ISF is representative of blood glucose and follows it rapidly when changes occur. The method consists of at least individual steps or groups of the following steps or from the overall sequence:

1. The point on the skin (in this case, the first region of the surface of the substance) is irradiated with a focused beam of a quantum cascade laser that may also be reflected at a mirror or concave mirror, and which is incrementally or continuously tuned over a specific infrared range in which radiation is absorbed glucose-specifically. Instead of the quantum cascade laser, a laser array having a plurality of lasers radiating with single wavelengths can also be used. The spectral range (or the individual wavelengths, typically 5 or more wavelengths) can be located between approximately 900 and approximately 1300 cm⁻¹, in which glucose has an absorption fingerprint, i.e. typical and representative absorption lines.
2. The excitation beam is used in a continuous mode (CW laser) or pulsed or modulated with a high pulse repetition rate. In addition, the excitation beam is modulated at low frequency, in particular in the frequency range between 10 and 1000 Hz. The low-frequency modulation can be performed with different periodic functions, in different embodiments with a sinusoid, a square wave or sawtooth wave or similar. A rectangular shape is the most advantageous according to the emission characteristic of a QCL.
3. By the irradiation of the skin, the IR radiation penetrates into the skin to a depth of about 50-100 μm and—depending on the wavelength—excites specific vibrations in the glucose molecule. These excitations from the vibration level v0 to v1 return to the ground state within a very short time; during this step heat is released.
4. As a result of the heat development according to (3), a thermal wave develops which propagates isotropically from the site of the absorption. Depending on the thermal diffusion length, determined by the low-frequency modulation described in (2), the thermal wave reaches the surface of the skin periodically at the modulation frequency.
5. The periodic appearance of the thermal wave on the surface corresponds to a periodic modulation of the heat radiation characteristic of the skin (surface of the sample substance). The skin can be described here approximately as a black-body radiator, the total emission of which by the Stefan-Boltzmann law is proportional to the fourth power of the surface temperature. With the measurement technique described in this document, the focus of the measurement is placed on the measurement of the heat conduction.
6. A detection device according to the patent claims of this application is used to detect the effect of a thermal and/or pressure wave arriving at the detection device on the refractive index of an optical waveguide device, in particular an interferometric device.
7. In the processing of the response signals, the modulation frequency can be specifically taken into account, for which purpose the response signal can be processed in a lock-in amplifier. By analysing the phase offset between the excitation signal and the heat radiation signal (response signal) by means of a control and processing device, the depth information can be obtained via the depth below the surface of the substance from which the response signals are predominantly received.
8. The depth information can also be obtained by selecting and analysing different low-frequency modulation frequencies for the excitation beam as described in (2) and combining the results for different modulation frequencies (wherein the results for different modulation frequencies can also be weighted differently). Differential methods, a quotient formation from at least two response signals in each case (for example, for a single wavelength and then passing by wavelengths through the measured spectrum) or other determination methods can be used to compensate for the absorption of the upper skin layers.
9. From the heat signal measured according to (6-8), which is dependent on the excitation wavelength, in one embodiment where glucose is to be detected the background is thus determined initially at non-glucose-relevant (or excluding glucose-relevant) wavelengths of the excitation beam, and then at (or including) glucose-relevant wavelengths the difference relative to the background signal. This results in the glucose concentration in the skin layer or skin layers, which is determined by the selected phase offset according to (7) or the different modulation frequencies according to (8) or their combination.

Although the invention has been illustrated and described in greater detail by means of preferred exemplary embodiments, the invention is not restricted by the examples disclosed and other variations can be derived therefrom by the person skilled in the art without departing from the scope of protection of the invention.

1. Device for analysing a substance, having:
a measuring body (1, 1a), which has a measuring surface (2) and is to be at least partially coupled with the substance (3) in the area of the measuring surface for measurement, in particular directly or by means of a medium, in particular a fluid, or is to be brought into contact with it directly or else by means of a medium,
a source of excitation radiation capable of generating light or an excitation beam of different wavelengths, in particular a laser device (4), in particular with a quantum cascade laser (QCL), a tuneable QCL, and/or with a laser array, preferably an array of QCLs, for generating one or more excitation beams (10) of different wavelengths, preferably in the infrared or medium-infrared spectral range, which is directed at the substance (3) when the measuring body (1, 1a) is coupled and/or in contact with the substance (3) in the region of the measuring surface (2), and
a detection device (5, 6, 7) which is at least partially integrated into or connected to the measuring body (1, 1a), comprising the following:
a source (5) for detection light, preferably coherent detection light (11), and
a first optical waveguide structure (6), which can be or is connected to the detection light source and which guides the detection light, the refractive index of which, at least in some sections, is dependent on the temperature and/or pressure, the first optical waveguide structure having at least one section (9) in which the light intensity depends on a phase shift of detection light in at least one part of the optical waveguide structure (6) due to a change in temperature or pressure.

2. Device according to claim 1, characterized in that at least one section of a projection of the first optical waveguide structure (6) in the direction of the surface normal of the measuring surface (2) is superimposed with said measuring surface (2).

3. Device according to claim 1 or 2, characterized in that a modulation device (8) is provided for modulating the intensity of the excitation beam (10).

4. Device according to claim 1, 2 or 3, characterized by a measuring device (7) for the direct or indirect detection of the light intensity in the first optical waveguide structure (6), in particular in a section (9) in which the light intensity depends on a phase shift of the detection light in at least one part of the first optical waveguide structure due to a change in temperature or pressure.

5. Device according to claim 1, 2, 3 or 4, characterized in that the detection device comprises an interferometric device, in particular an interferometer (12) and/or an optical waveguide resonance element, in particular a resonance ring (13) or a resonance plate.

6. Device according to any one of claims 1 to 5, characterized in that the first optical waveguide structure (6), in particular an interferometric device of the first optical waveguide structure, comprises at least one fibre-optic optical waveguide (14), which is connected to the measuring body (1) at least in some sections.

7. Device according to any one of claims 1 to 6, characterized in that an optical waveguide (15, 16) of the first optical waveguide structure (6), in particular of an interferometric device of the first optical waveguide structure, is integrated in a substrate (1a) of the measuring body or is connected to a substrate, the first optical waveguide structure (6) having at least one silicon optical waveguide, which is connected to an insulating substrate or is integrated into an insulating substrate, and in particular the silicon optical waveguide also being at least partially covered by an insulator, in particular SiO₂.

8. Device according to any one of the preceding claims, characterized in that the excitation beam (1o), in particular in the region of the measuring surface of the measuring body or a region adjacent to the measuring surface (2), passes through the material of the measuring body (1, 1a) or a region adjacent to the measuring surface, wherein the measuring body or the region penetrated by the excitation beam (1o) is transparent to the excitation beam.

9. Device according to any one of the preceding claims, characterized in that the excitation beam (1o) is guided inside the measuring body (1, 1a) or along the measuring body by means of a second optical waveguide structure (17).

10. Device according to any one of the preceding claims, characterized in that the excitation beam (1o) between the laser device (4) and the substance (3) to be analysed passes through a continuous opening (18) of the measuring body (1, 1a), wherein the opening ends in particular at a distance in front of the measuring surface or penetrates the measuring surface (2) or is arranged in a region which is directly adjacent to the measuring surface and/or adjoins it.

11. Device according to any one of the preceding claims, characterized in that the measuring body (1, 1a) is formed as a flat body, in particular as a plane-parallel body in the form of a plate, wherein in particular the thickness of the measuring body in the direction perpendicular to the measuring surface (2) is less than 50% of the smallest extension of the measuring body in a direction extending in the measuring surface, in particular, less than 25%, more particularly less than 10%.

12. Device according to any one of the preceding claims, characterized in that the measuring body (1, 1a) comprises or carries a mirror device (19) for reflecting the excitation beam (1o) irradiated by the laser device (4) onto the measuring surface (2).

13. Device according to any one of the preceding claims, characterized in that the excitation beam (1o) is oriented into the measuring body (1, 1a) parallel to the measuring surface (2) or at an angle of less than 30 degrees, in particular less than 20 degrees, more particularly less than 10 degrees or less than 5 degrees to the measuring surface, and that the excitation beam is diverted or deflected towards the measuring surface, wherein the excitation beam in particular passes through the measuring surface or an imaginary continuation of the measuring surface in the region of a continuous opening (18) in the measuring body.

14. Device according to any one of the preceding claims, characterized in that in the measuring body (1, 1a), behind and/or next to the detection device (5, 6, 7) viewed from the measuring surface (2), in particular behind and/or next to the first optical waveguide structure (6), in particular adjacent to and in thermal contact with the latter, at least one heat sink (20) is arranged in the form of a solid body or material, wherein in particular, the specific thermal capacity and/or specific thermal conductivity of the body or the material of the heat sink is greater than the specific thermal capacity and/or thermal conductivity of the material of the detection device (5, 6, 7) and/or of the first optical waveguide structure and/or the substrate (1a) of the first optical waveguide structure (6) and/or of the other materials which comprise the measuring body (1, 1a) and/or that a barrier (30, 40, 41) is provided in the measuring body (1, 1a), which at least partially shields a part of the detection device, in particular a part of the first optical waveguide structure (6), more particularly a reference arm of an interferometer, from the effect of the thermal and/or pressure wave and/or that the first optical waveguide structure (6) of the detection device comprises at least two measuring sections (15a, 16a), arranged in particular on different arms of an interferometer and in which the refractive index changes as a function of pressure and/or temperature changes, in particular of a pressure and/or thermal wave, so that a phase shift occurs in the detection light passing through the measuring sections followed by a resulting intensity change in the detection light in a further section as a function of pressure and/or temperature changes, the two measuring sections being arranged in the measuring body in such a way that they are passed through by a pressure and/or thermal wave, which propagates through the measuring body starting from the measuring surface (2), in particular from the region of the measuring surface in which the excitation beam penetrates it, one after the other, in particular in time intervals temporally shifted relative to one another or with a time delay.

15. Sensor, in particular for a device according to any one of the preceding claims, having a measuring body (1, 1a) which has a measuring surface (2) and is to be at least partially coupled with, in particular brought into contact with, a substance (3) in the region of the measuring surface for measuring a temperature and/or pressure wave, and having a detection device (5, 6, 7) which is at least partially integrated into or connected to the measuring body (1, 1a), comprising the following:
a source (5) for coherent detection light (11), and
a first optical waveguide structure (6), which can be connected or is connected to the source for the detection light and which guides the detection light, the refractive index of which at least in sections is dependent on the temperature and/or pressure,
at least one section (9) in which the light intensity depends on a phase shift of the detection light in at least one part of the first optical waveguide structure (6) due to a change in temperature or pressure, the first optical waveguide structure having an interferometric device, in particular an interferometer (12) and/or an optical waveguide resonance ring (13) or another optical waveguide resonance element, and
a measuring device (7) for detecting the light intensity in or of the interferometric device.

16. Method for operating a device according to any one of the preceding claims, characterized in that a modulated excitation beam (1o) is directed, in particular through the measuring body, onto the substance (3) to be analysed and that a temporal light intensity profile or waveform or a periodic light intensity change is detected by the detection device, these being detected for a plurality of wavelengths of the excitation beam by measuring the light intensity change in the first optical waveguide structure or by measuring the light intensity of light emitted from the first optical waveguide structure and obtaining an absorption spectrum of the substance to be analysed from the acquired data.

17. Method according to claim 16, characterized in that the measurement is carried out for different modulation frequencies of the excitation beam (1o) and that a corrected absorption spectrum is determined from the combination of absorption spectra obtained.
